(19) United States
(12) Patent Application Publication
(10) Pub. No.: US 2023/0346826 A1
(43) Pub. Date: Nov. 2, 2023

(54) COMPOSITIONS AND METHODS FOR TREATING AND/OR PREVENTING AUTOIMMUNE DISORDERS

(71) Applicant: The Regents of the University of Michigan, Ann Arbor, MI (US)

(72) Inventors: James J. Moon, Ann Arbor, MI (US); Kai Han, Ann Arbor, MI (US); Fang Xie, Ann Arbor, MI (US); Xingwu ZHOU, Ann Arbor, MI (US)

(21) Appl. No.: 18/027,763

(22) PCT Filed: Sep. 23, 2021
(86) PCT No.: PCT/US2021/051628

§ 371 Date: Mar. 22, 2023

Related U.S. Application Data
(60) Provisional application No. 63/082,236, filed on Sep. 23, 2020.

(51) Int. Cl. A61P 37/06; A61K 47/36; A61K 31/733; A61K 9/00

(52) U.S. Cl. CPC A61K 31/733; A61K 9/0053; A61K 47/36; A61P 37/06

(57) ABSTRACT

This invention relates generally to compositions comprising a gel-based inulin formulation associated with (e.g., complexed, conjugated, encapsulated, absorbed, adsorbed, admixed) one or more therapeutic agents (e.g., immunomodulatory agent, immunosuppressant, allergen) and related methods for the treatment of autoimmune disorders (e.g., colitis) (e.g, allergy, such as food allergy). Also provided herein are compositions and methods for modulating an immune response associated with an autoimmune disorder (e.g., allergy) and/or inducing immune tolerance or desensitization to an autoimmune disorder (e.g., allergy, such as a food allergy).

### CROSS REFERENCE TO RELATED APPLICATION

This application claims priority to U.S. Provisional Pat. Application Serial No. 63/082,236 filed Sep. 23, 2020, which is incorporated herein by reference in its entirety.

### FEDERALLY SPONSORED RESEARCH OR DEVELOPMENT

This invention was made with government support under DK125087 awarded by the National Institutes of Health. The government has certain rights in this invention.

### FIELD OF THE INVENTION

This invention relates generally to compositions comprising a gel-based inulin formulation associated with (e.g., complexed, conjugated, encapsulated, absorbed, adsorbed, admixed) one or more therapeutic agents (e.g., immunomodulatory agent, immunosuppressant, allergen) and related methods for the treatment of autoimmune disorders (e.g., colitis) (e.g, allergy, such as food allergy). Also provided herein are compositions and methods for modulating an immune response associated with an autoimmune disorder (e.g., allergy) and/or inducing immune tolerance or desensitization to an autoimmune disorder (e.g., allergy, such as a food allergy).

### BACKGROUND OF THE INVENTION

Food allergy is a prevalent disease around the world. Patients suffering from food allergy will show anaphylactic reactions including itching, diarrhea, or even life-threatening loss of consciousness after unconscious exposure to allergens (see, R. Khamsi. Food allergies: the psychological toll. Nature 2020, 588, S4-S6). Currently available interventions or therapies are limited to allergen avoidance and emergency treatments. The U.S. Food and Drug Administration (FDA) recently approved the first drug Palforzia showing promising market prospect for peanut allergy therapy (see, A. Mullard. FDA approves first peanut allergy drug. Nat Rev Drug Discovery 2020, 19, 156). As an oral immunotherapy (OIT) strategy, Palforzia consists of initial dose escalation, up-dosing, and maintenance phases. However, there are many limitations of Palforzia. 12.9% of patients in the clinical trial of Palforzia discontinued the therapy due to adverse events during the first 6 months (see, T. Casale, A. Wesley Burks, J. Baker, et al. Safety of Peanut (Arachis Hypogaea) Allergen Powder-dnfp in Children and Teenagers With Peanut Allergy: Pooled Analysis From Controlled and Open-Label Phase 3 Trials. J Allergy Clin Immunol 2021,147, AB106). Therefore, there is an urgent need for new approaches.

As an alternative, oral administration of probiotics or faecal microbiota transplantation (FMT) has been explored for allergy therapy, partly due to the beneficial commensal microorganism-induced generation of immune tolerance cells in gastrointestinal (GI) tract (see, A. Abdel-Gadir, E. Stephen-Victor, G. K. Gerber, et al. Microbiota therapy acts via a regulatory T cell MyD88/RORyt pathway to suppress food allergy. Nat Med 2019, 25,1164-1174). However, the precise definition of beneficial commensal microorganisms for allergy patients is not known, and there are concerns of opportunistic infection in FMT.

Thus, there is an urgent need to develop new therapeutics for long-term control of autoimmune disorders such as peanut allergy as well as other food allergies.

The present invention addresses this urgent need.

### SUMMARY

Experiments described during the course of developing embodiments for the present invention report a dietary fiber hydrogel-based OIT strategy for efficient gut microbiota modulation, decreasing level of allergen-specific IgE, and the induction of regulatory T (Treg) cells, thereby realizing protection effects in multiple food allergy models.

Accordingly, the present invention relates generally to compositions comprising a gel-based inulin formulation associated with (e.g., complexed, conjugated, encapsulated, absorbed, adsorbed, admixed) one or more therapeutic agents (e.g., immunomodulatory agent, immunosuppressant, allergen) and related methods for the treatment of autoimmune disorders (e.g., colitis) (e.g, allergy, such as food allergy). Also provided herein are compositions and methods for modulating an immune response associated with an autoimmune disorder (e.g., allergy) and/or inducing immune tolerance or desensitization to an autoimmune disorder (e.g., allergy, such as a food allergy).

In certain embodiments, the present invention provides a composition comprising a gel-based inulin formulation associated with one or more therapeutic agents. Such compositions are not limited to a particular meaning of associated. In some embodiments, associated with comprises one or more of the following: complexed, conjugated, encapsulated, absorbed, adsorbed, and admixed.

In some embodiments, the gel-based inulin formulation has an average degree of polymerization at or higher than 20 and at or less than 47. In some embodiments, the gel-based inulin formulation has an average degree of polymerization of approximately 26 (e.g., 20, 21, 22, 23, 24, 25, 26, 27, 28, 29, 30, 31, 32).

In some embodiments, the gel-based inulin formulation further comprises one or more prebiotic compounds selected from a fructo-oligosaccharide, a short-chain fructo-oligosaccharide, an isomalt-oligosaccharide, a transgalacto-oligosaccharide, a pectin, a xylo-oligosaccharide, a chitosan-oligosaccharide, a beta-glucan, an arable gum modified starch, a resistant potato starch, guar gum, bean gum, gelatin, glycerol, a polydextrose, a D-tagatose, an acacia fiber, carob, an oat, and a citrus fiber.

Such compositions are not limited to a particular type or kind of therapeutic agent.

In some embodiments, the therapeutic agent is one or more allergens.

In some embodiments, the or more allergens are independently selected from an allergen source selected from animal products, plants, food, insect stings, drugs, fungal spores, and microorganisms.

In some embodiments, the one or more allergens are independently selected from an animal product allergen, plant allergens, insect sting allergens, drug allergens, fungal allergens, microorganism allergens.

In some embodiments, the one or more allergens are independently selected from abalone (perlemoen), acerola, Alaska pollock, almond, aniseed, apple, apricot, avocado, banana, barley, bell pepper, Brazil nut, buckwheat, cabbage, carp, carrot, cashew, caster bean, celery, celeriac, cherry, chestnut, chickpea (garbanzo, bengal gram), cococa, coconut, cod, cotton seed, courgett (zucchini), crab, date, egg, fig, fish, flax seed (linseed), frog, garden plum, garlic, grape, hazelnut, kiwi fruit (Chinese gooseberry), lentil, lettuce, lobster, lupin (lupine), lychee, mackerel, maize (corn), mango, melon, milk, mustard, oat oyster, peach, peanut (ground nuts, monkey nuts), pear, pecan, persimmon, pine nut, pineapple, pomegranate, poppy seed, potato, pumpkin, rice, rye, salmon, sesame, sesame seed, shrimp (black tiger shrimp, brown shrimp, greasyback shrimp, Indian prawn, Neptune rose shrimp, white shrimp), snail, soybean (soya), squid, strawberry, sunflower seed, tomato, tuna, turnip, walnut, and wheat (bread-making wheat, pasta wheat, kamut, spelt).

In some embodiments, the one or more allergens are independently selected from
animal products including fur, dander, cockroach calyx, wool, dust mite excretion, and fel d 1 (e.g., a protein produced in cat saliva and sebaceous glands);
allergens from plant include plant pollens from grass such as ryegrass; weeds such as ragweed, nettle, sorrel; and trees such as birch, alder, hazel, oak, elm, and maple;
allergens from insect stings include bee sting, wasp sting, and mosquito stings;
drug allergens including penicillin, sulfonamides, quinidine, phenylbutazone, thiouracils, methyldopa, hydantoins, and salicytates;
fungal allergens include basidiospores such as Ganoderma; mushroom spores; allergens from the aspergillus and alternaria-penicillin families; and cladosporium spores;
allergens from microorganisms that can cause an allergic reaction include viruses and bacteria; and
food allergens including peanuts, tree nuts such as pecans and almonds, eggs, milk, shellfish, fish, wheat and their derivative, soy and their derivatives.

In some embodiments, one or more allergens is two or more allergens (e.g., 2, 3, 4, 5, 10, 20, 50, 100, 1,000).

In some embodiments wherein the therapeutic agent is an allergen, upon administration of the composition in a therapeutically effective amount to a subject the composition is capable of one or more of:
treating an allergy (e.g., a food allergy),
reducing one or more symptom associated with an allergy (e.g., food allergy),
modulating one or more immune responses associated with an allergy (e.g., food allergy),
inducing the proliferation and/or accumulation of regulatory T cells in the subject,
suppressing the production of IgE antibodies (e.g., total IgE antibodies or allergen-specific IgE antibodies),
suppressing one or more Th2 immune responses, and
allowing the subject to survive a challenge with the allergen (e.g., in case of an anaphylactic allergic response in the inadvertent exposure to a peanut allergen).

In some embodiments, the therapeutic agent is at least one immunomodulatory agent selected from fingolimod; 2-(1′H-indole-3′-carbonyl)-thiazole-4-carboxylic acid methyl ester (ITE) or related ligands; Trichostatin A; Suberoylanilide hydroxamic acid (SAHA); statins; mTOR inhibitors; TGF-β signaling agents; TGF-β receptor agonists; histone deacetylase inhibitors; corticosteroids; inhibitors of mitochondrial function; NF-κβ inhibitors; adenosine receptor agonists; prostaglandin E2 agonists (PGE2; phosphodiesterase inhibitors; proteasome inhibitors; kinase inhibitors; G-protein coupled receptor agonists; G-protein coupled receptor antagonists; glucocorticoids; retinoids; cytokine inhibitors; cytokine receptor inhibitors; cytokine receptor activators; peroxisome proliferator-activated receptor antagonists; peroxisome proliferator-activated receptor agonists; histone deacetylase inhibitors; calcineurin inhibitors; phosphatase inhibitors; PI3 KB inhibitors; autophagy inhibitors; aryl hydrocarbon receptor inhibitors; proteasome inhibitor I (PSI); oxidized ATPs IDO; vitamin D3; cyclosporins; aryl hydrocarbon receptor inhibitors; resveratrol; azathiopurine (Aza); 6-mercaptopurine (6-MP); 6-thioguanine (6-TG); FK506; sanglifehrin A; salmeterol; mycophenolate mofetil (MMF); aspirin and other COX inhibitors; niflumic acid; estriol; triptolide; OPN-305, OPN-401; Eritoran (E5564); TAK-242; Cpn10; NI-0101; 1A6; AV411; IRS-954 (DV-1079); IMO-3100; CPG-52363; CPG-52364; OPN-305; ATNC05; NI-0101; IMO-8400; Hydroxychloroquine; CU-CPT22; C29; Ortho-vanillin; SSL3 protein; OPN-305; 5 SsnB; Vizantin; (+)-N-phenethylnoroxymorphone; VB3323; Monosaccharide 3; (+)-Naltrexone and (+)-naloxone; HT52; HTB2; Compound 4a; CNTO2424; TH1020; INH-ODN; E6446; AT791; CpG ODN 2088; ODN TTAGGG; COV08-0064; 2R9; GpG oligonucleotides; 2-aminopurine; Amlexanox; Bay11-7082; BX795; CH-223191; Chloroquine; CLI-095; CU-CPT9a; Cyclosporin A; CTY387; Gefitnib; Glybenclamide; H-89; H-131; Isoliquiritigenin; MCC950; MRT67307; OxPAPC; Parthenolide; Pepinh-MYD; Pepinh-TRIF; Polymyxin B; R406; RU.521; VX-765; YM201636; Z-VAD-FMK; and AHR-specific ligands; including but not limited to 2,3,7,8-tetrachloro-dibenzo-p-dioxin (TCDD); tryptamine (TA); and 6 formylindolo[3,2 b]carbazole (FICZ)).

In particular embodiments, the therapeutic agent is an immunosuppressant selected from fingolimod; 2-(1′H-indole-3′-carbonyl)-thiazole-4-carboxylic acid methyl ester (ITE) or related ligands; Trichostatin A; and/or Suberoylanilide hydroxamic acid (SAHA).

In some embodiments, the gel-based inulin formulation is further admixed with an adjuvant (e.g., CPG, polyIC, poly-ICLC, 1018 ISS, aluminum salts, Amplivax, AS15, BCG, CP-870,893, CpG7909, CyaA, dSLIM, GM-CSF, IC30, IC31, Imiquimod, ImuFact IMP321, IS Patch, ISS, ISCOMATRIX, Juvlmmune, LipoVac, MF59, monophosphoryl lipid A, Montanide IMS 1312, Montanide ISA 206, Montanide ISA 50V, Montanide ISA-51, OK-432, OM-174, OM-197-MP-EC, ONTAK, PepTel.RTM, vector system, PLGA microparticles, imiquimod, resiquimod, gardiquimod, 3M-052, SRL172, Virosomes and other Virus-like particles, YF-17D, VEGF trap, beta-glucan, Pam3Cys, Aquila’s QS21 stimulon, vadimezan, AsA404 (DMXAA), and any derivative of an adjuvant).

In some embodiments, the composition does not contain an adjuvant.

In certain embodiments, the present invention provides a method of treating, preventing and/or ameliorating symptoms of an autoimmune disorder, comprising administering to a subject (e.g., a human subject) (e.g., a mammalian subject) in need thereof a therapeutically effective amount of such a composition (e.g., a composition comprising a gel-based inulin formulation associated with one or more therapeutic agents (e.g., immunomodulatory agent, immunosuppressant, allergen) (e.g., a composition comprising a gel-based inulin formulation associated with one or more therapeutic agents (e.g., immunomodulatory agent, immunosuppressant, allergen) and a prebiotic compound) (e.g., a composition comprising a gel-based inulin formulation associated with one or more therapeutic agents. In some embodiments, the autoimmune disorder is selected from allergic asthma, allergic colitis, animal allergies, atopic allergies, hay fever, skin allergy, hives, atopic dermatitis, anaphylaxis, allergic rhinitis, drug or medicinal allergy, eczema (atopic dermatitis), food allergy, fungal allergy, insect allergy (including insect bite/venom allergies), mold allergies, plant allergies, and pollenosis.

In some embodiments, the administering results in the suppression of an immune response associated with a food allergy.

In some embodiments, the administering results in the suppression of the production of IgE antibodies.

In some embodiments, the administering results in the suppression of a Th2 immune response.

In some embodiments, the allergy is a good allergy selected from the group consisting of a nut allergy, a fish allergy, a wheat allergy, a milk allergy, a peanut allergy, a tree nut allergy, a shellfish allergy, a soy allergy, a seed allergy, a sesame seed allergy, and an egg allergy.

In some embodiments, the allergy is a peanut allergy.

In certain embodiments, the present invention provides kits comprising such compositions (e.g., a composition comprising a gel-based inulin formulation associated with one or more therapeutic agents (e.g., immunomodulatory agent, immunosuppressant, allergen)) and instructions for administering such compositions to a subject.

### BRIEF DESCRIPTION OF THE DRAWINGS

FIGS. 1A-G: Inulin gel plus OVA combo-therapy protect against OVA allergy. a, Image of inulin hydrogel. b, Therapeutic regimen. BABL/c mice were sensitized with OVA/Alum on days 0 and 14. From day 29, mice were orally gavaged with PBS, OVA (1 mg per dose), and inulin gel (55 mg per dose) plus OVA (1 mg per dose), respectively. From day 49, mice were orally challenged with OVA (50 mg/dose) in an alternative days for 6 times. c, Body temperature drop at the 6^{th} time of challenge, d, anaphylactic symptom score during challenge, e, body weight drop between the 1^{st} and 6^{th} time of challenge, f, OVA-specific IgE level in serum on days 48, 55, and 61, respectively. g, Number of mast cells and the representative microscopy images of toluidine blue-stained histological sections from the jejunum. Arrows indicate mast cells. Data represent the mean ± s.e.m. *P < 0.01, **P < 0.01, ****P < 0.0001. Data were analysed using a two-way ANOVA (c-e) or one-way ANOVA (f,h), or unpaired Student’s t-test (g). # in c indicates statistically significant differences between inulin gel/OVA vs. OVA.

FIGS. 2A-G: Inulin gel plus OVA (escalated dose) combo-therapy protect against OVA allergy. a, Therapeutic regimen. BABL/c mice were sensitized with OVA/Alum for 2 times. From day 29, mice were orally gavaged with OVA (in an escalated dose manner) and inulin gel (55 mg per dose) plus OVA (in an escalated dose manner), respectively. From day 49, mice were orally challenged with OVA (50 mg/dose) in an alternative days for 6 times. b, Diarrhea occurrence and score, c, body weight drop between before and after the 3^{rd} time challenge, d, anaphylactic symptom score, e, MMCP-1, f, Number of mast cells and the representative microscopy images of toluidine blue-stained histological sections from the jejunum on day 62. Arrows indicate mast cells. g, Splenocytes were restimulated with 250 µg/mL OVA on day 62. After 72 h incubation, the supernatant was collected for cytokines analysis. Data represent the mean ± s.e.m. *P < 0.01, **P < 0.01, ***P < 0.001, ****P < 0.0001. Data were analysed using a two-way ANOVA (b,d) or unpaired Student’s t-test (c,e-g).

FIGS. 3A-C: Inulin gel OVA combo-therapy protect against OVA allergy. BALB/c mice were treated as shown in FIG. 1b. Shown are the (a) number of Foxp3⁺CD4⁺ Tregs in ileum and the representative flow cytometry plots, b, number of CD103⁺ DC cell in spleen, c, frequencies of LAG3⁺CD49b⁺ Tr1 cells in spleen and MLN on day 61. Data represent the mean ± s.e.m. *P < 0.01. Data were analysed using a two-way ANOVA.

FIGS. 4A-D: Inulin gel plus PE combo-therapy protect against peanut allergy. a, Therapeutic regimen. C3H/HeJ mice were kept on a normal chow diet and sensitized with PE/CT for 4 weeks. From day 36, mice were orally gavaged with PBS, PE (1.2 mg per dose), inulin gel (45 mg per dose), and inulin gel (45 mg per dose) plus PE (1.2 mg per dose), respectively. Mice received OIT treatment 4 times per week for 4 weeks, following with i.p. injection of PE (125 µg/dose) on day 73. b-d, Average body temperature drop (b), individual body temperature drop (c) and anaphylactic symptoms score (d). Data represent the mean ± s.e.m. *P < 0.01, **P < 0.01, ****P < 0.0001. Data were analysed using a two-way ANOVA (b) or one-way ANOVA with Bonferroni’s multiple comparisons test (d).

FIGS. 5A-E: C57BL/6 mice were provided with 3% DSS-containing water for 6 days. From 0 d to 7 d, mice were orally administered with PBS, 1 mg free IALD, or 1 mg IALD mixed within 60 mg Inulin gel. B. Daily bodyweight changes in each group for 9 d. C-E. On day 9, animals were euthanized and colon were collected (C), colon length (D) and cecum weight (E) were measured.

### DEFINITIONS

As used herein, the term “about” is used herein to mean a value that is ±10% of the recited value.

As used herein, the term “absorbed” refers to an allergen that is taken into and stably retained in the interior, that is, internal to the outer surface, of gel-based inulin formulation.

As used herein, by “administering” is meant a method of giving a dosage of a composition described herein (e.g., gel-based inulin formulations associated with one or more allergens) (e.g., a gel-based inulin formulation associated with one or more allergens and one or both of a prebiotic compound and a therapeutic agent) to a subject. The compositions utilized in the methods described herein can be administered by any suitable route, including, for example, by inhalation, nebulization, aerosolization, intranasally, intratracheally, intrabronchially, orally, parenterally (e.g., intravenously, subcutaneously, or intramuscularly), nasally, rectally, topically, or buccally. The compositions utilized in the methods described herein can also be administered locally or systemically. The preferred method of administration can vary depending on various factors (e.g., the components of the composition being administered, and the severity of the condition being treated).

As used herein, the term “admixed” refers to an allergen that is dissolved, dispersed, or suspended in a gel-based inulin formulation. In some cases, the allergen may be uniformly admixed in the gel-based inulin formulation.

As used herein, the term “adsorbed” refers to the attachment of an allergen to the external surface of a gel-based inulin formulation. Such adsorption preferably occurs by electrostatic attraction. Electrostatic attraction is the attraction or bonding generated between two or more oppositely charged or ionic chemical groups. Generally, the adsorption is typically reversible.

As used herein, the term “allergen” refers to a compound, substance or composition that causes, elicits or triggers and immune reaction (e.g., allergic reaction) in a subject. As such, allergens are typically referred to as antigens. An allergen is typically a protein or a polypeptide.

As used herein, the term “allergy” refers to a disorder (or improper reaction) of the immune system often also referred to as atopy. Allergic reactions occur to normally harmless environmental substances known as allergens; these reactions are acquired, predictable, and rapid. Strictly, allergy is one of four forms of hypersensitivity and is called type I (or immediate) hypersensitivity. It is characterized by excessive activation of certain white blood cells called mast cells and basophils by a type of antibody known as IgE, resulting in an extreme inflammatory response. Common allergic reactions include eczema, hives, hay fever, asthma, food allergies, and reactions to the venom of stinging insects such as wasps and bees. Mild allergies like hay fever are highly prevalent in the human population and cause symptoms such as allergic conjunctivitis, itchiness, and runny nose.

As used herein, the terms “autoimmune disorder” and “autoimmune disease”, used herein interchangeably, refers to a medical condition in which a subject’s immune system mistakenly attacks the subject’s own body.

As used herein, a “combination therapy” or “administered in combination” means that two or more (e.g., 2, 3, 4, 5, 6, 7, 8, 9, 10 or more) different agents or treatments are administered to a subject as part of a defined treatment regimen for a particular disease or condition (e.g., an autoimmune disorder). The treatment regimen defines the doses and periodicity of administration of each agent such that the effects of the separate agents on the subject overlap. In some embodiments, the delivery of the two or more agents is simultaneous or concurrent and the agents may be co-formulated. In some embodiments, the two or more agents are not co-formulated and are administered in a sequential manner as part of a prescribed regimen. In some embodiments, administration of two or more agents or treatments in combination is such that the reduction in a symptom, or other parameter related to the disorder, is greater than what would be observed with one agent or treatment delivered alone or in the absence of the other. The effect of the two treatments can be partially additive, wholly additive, or greater than additive (e.g., synergistic). Sequential or substantially simultaneous administration of each therapeutic agent can be affected by any appropriate route including, but not limited to, by inhalation, nebulization, aerosolization, intranasally, intratracheally, intrabronchially, orally, parenterally (e.g., intravenously, subcutaneously, or intramuscularly), orally, nasally, rectally, topically, buccally, or by direct absorption through mucous membrane tissues. The therapeutic agents can be administered by the same route or by different routes. For example, a first therapeutic agent of the combination may be administered by intravenous injection while a second therapeutic agent of the combination may be administered orally.

As used herein, the term “complexed” as used herein relates to the non-covalent interaction of an allergen with a gel-based inulin formulation.

As used herein, the term “conjugated” as used herein indicates a covalent bond association between an allergen and a gel-based inulin formulation.

As used herein, the term “drug” or “therapeutic agent” is meant to include any molecule, molecular complex, or substance administered to an organism for diagnostic or therapeutic purposes.

As used herein, the term “immunomodulatory agent” refers to a compound that stimulates or suppresses the immune system. As used herein, the term “immunosuppressant” refers to a compound that causes antigen presenting cells (APCs) to have an immunosuppressive (e.g., tolerogenic effect). An immunosuppressive effect generally refers to the production or expression of cytokines or other factors by the APC that reduces, inhibits or prevents an undesired immune response or that promotes a desired immune response. When the APC results in an immunosuppressive effect on immune cells that recognize an antigen presented by the APC, the immunosuppressive effect is said to be specific to the presented antigen. Such effect is also referred to herein as a tolerogenic effect. Without being bound by any particular theory, it is thought that the immunosuppressive or tolerogenic effect is a result of the immunosuppressant being delivered to the APC, preferably in the presence of an antigen (e.g., an administered antigen or one that is already present in vivo). Accordingly, the immunosuppressant includes compounds that provide a tolerogenic immune response to an antigen that may or may not be provided in the same composition or a different composition. In one embodiment, the immunosuppressant is one that causes an APC to promote a regulatory phenotype in one or more immune effector cells. For example, the regulatory phenotype may be characterized by the inhibition of the production, induction, stimulation or recruitment of antigen-specific CD8+ T cells, the production, induction, stimulation or recruitment of Treg cells, etc. This may be the result of the conversion of CD8+ T cells or B cells to a regulatory phenotype. This may also be the result of induction of FoxP3 in other immune cells, such as CD4+ T cells, macrophages and iNKT cells. In one embodiment, the immunosuppressant is one that affects the response of the APC after it processes an antigen. In another embodiment, the immunosuppressant is not one that interferes with the processing of the antigen. In a further embodiment, the immunosuppressant is not an apoptotic-signaling molecule. In another embodiment, the immunosuppressant is not a phospholipid.

Immunomodulatory agents include, but are not limited to, statins; mTOR inhibitors, such as rapamycin or a rapamycin analog; TGF-β signaling agents; TGF-β receptor agonists; histone deacetylase inhibitors, such as Trichostatin A; corticosteroids; inhibitors of mitochondrial function, such as rotenone; P38 inhibitors; NF-κβ inhibitors, such as 6Bio, Dexamethasone, TCPA-1, IKK VII; adenosine receptor agonists; prostaglandin E2 agonists (PGE2), such as Misoprostol; phosphodiesterase inhibitors, such as phosphodiesterase 4 inhibitor (PDE4), such as Rolipram; proteasome inhibitors; kinase inhibitors; G-protein coupled receptor agonists; G-protein coupled receptor antagonists; glucocorticoids; retinoids; cytokine inhibitors; cytokine receptor inhibitors; cytokine receptor activators; peroxisome proliferator-activated receptor antagonists; peroxisome proliferator-activated receptor agonists; histone deacetylase inhibitors; calcineurin inhibitors; phosphatase inhibitors; PI3 KB inhibitors, such as TGX-221; autophagy inhibitors, such as 3-Methyladenine; aryl hydrocarbon receptor inhibitors; proteasome inhibitor I (PSI); and oxidized ATPs, such as P2X receptor blockers. Immunosuppressants also include IDO, vitamin D3, cyclosporins, such as cyclosporine A, aryl hydrocarbon receptor inhibitors, resveratrol, azathiopurine (Aza), 6-mercaptopurine (6-MP), 6-thioguanine (6-TG), FK506, sanglifehrin A, salmeterol, mycophenolate mofetil (MMF), aspirin and other COX inhibitors, niflumic acid, estriol; triptolide; OPN-305, OPN-401; Eritoran (E5564); TAK-242; Cpn10; NI-0101; 1A6; AV411; IRS-954 (DV-1079); IMO-3100; CPG-52363; CPG-52364; OPN-305; ATNC05; NI-0101; IMO-8400; Hydroxychloroquine; CU-CPT22; C29; Ortho-vanillin; SSL3 protein; OPN-305; 5 SsnB; Vizantin; (+)-N-phenethylnoroxymorphone; VB3323; Monosaccharide 3; (+)-Naltrexone and (+)-naloxone; HT52; HTB2; Compound 4a; CNTO2424; TH1020; INH-ODN; E6446; AT791; CpG ODN 2088; ODN TTAGGG; COV08-0064; 2R9; GpG oligonucleotides; 2-aminopurine; Amlexanox; Bay1 1-7082; BX795; CH-223191; Chloroquine; CLI-095; CU-CPT9a; Cyclosporin A; CTY387; Gefitnib; Glybenclamide; H-89; H-131; Isoliquiritigenin; MCC950; MRT67307; OxPAPC; Parthenolide; Pepinh-MYD; Pepinh-TRIF; Polymyxin B; R406; RU.521; VX-765; YM201636; Z-VAD-FMK; and AHR-specific ligands; including but not limited to 2,3,7,8-tetrachloro-dibenzo-p-dioxin (TCDD); tryptamine (TA); and 6 formylindolo[3,2 b]carbazole (FICZ). In particular embodiments, the immunosuppressant is FTY720 (also known as fingolimod) (Chung and Harung, Clin. Neuropharmacol 33: 91-101, 2010), AhR activation by 2-(1′H-indole-3′-carbonyl)-thiazole-4-carboxylic acid methyl ester (ITE) or related ligands (Yeste A, et al. Proc. Natl. Acad. Sci. USA 109: 11270-11275, 2012; Quintana F.J., et al Proc. Natl. Acad. Sci. USA 107: 20768-20773, 2010), Trichostatin A (TSA) (Reilly C.M. et al. J. Autoimmun 31: 123-130. 2008). Suberoylanilide hydroxamic acid (SAHA), a histone deacetylase inhibitor, (Lucas J.L., et al. Cell Immunol 257: 97-104, 2009) and/or Rapamycin (Rapa) (Maldonado, R.A., et al Proc. Natl. Acad. Sci. USA 112:E156-165, 2015). In embodiments, the immunosuppressant may comprise any of the agents provided herein.

The immunosuppressant can be a compound that directly provides the immunosuppressive (e.g., tolerogenic) effect on APCs or it can be a compound that provides the immunosuppressive (e.g., tolerogenic) effect indirectly (i.e., after being processed in some way after administration). Immunosuppressants, therefore, include prodrug forms of any of the compounds provided herein.

Immunosuppressants also include nucleic acids that encode the peptides, polypeptides or proteins provided herein that result in an immunosuppressive (e.g., tolerogenic) immune response. In embodiments, therefore, the immunosuppressant is a nucleic acid that encodes a peptide, polypeptide or protein that results in an immunosuppressive (e.g., tolerogenic) immune response, and it is the nucleic acid that is coupled to the gel-based inulin formulation.

The nucleic acid may be DNA or RNA, such as mRNA. In embodiments, the compositions comprise a complement, such as a full-length complement, or a degenerate (due to degeneracy of the genetic code) of any of the nucleic acids provided herein. In embodiments, the nucleic acid is an expression vector that can be transcribed when transfected into a cell line. In embodiments, the expression vector may comprise a plasmid, retrovirus, or an adenovirus amongst others. Nucleic acids can be isolated or synthesized using standard molecular biology approaches, for example by using a polymerase chain reaction to produce a nucleic acid fragment, which is then purified and cloned into an expression vector. Additional techniques useful in the practice of this invention may be found in Current Protocols in Molecular Biology 2007 by John Wiley and Sons, Inc.; Molecular Cloning: A Laboratory Manual (Third Edition) Joseph Sambrook, Peter MacCallum Cancer Institute, Melbourne, Australia; David Russell, University of Texas Southwestern Medical Center, Dallas, Cold Spring Harbor.

Other exemplary immunosuppressants include, but are not limited, small molecule drugs, natural products, antibodies (e.g., antibodies against CD20, CD3, CD4), biologics-based drugs, carbohydrate-based drugs, nanoparticles, liposomes, RNAi, antisense nucleic acids, aptamers, methotrexate, NSAIDs; fingolimod; natalizumab; alemtuzumab; anti-CD3; tacrolimus (FK506), etc. Further immunosuppressants, are known to those of skill in the art, and the invention is not limited in this respect.

As used herein, the term “in vitro” refers to an artificial environment and to processes or reactions that occur within an artificial environment. In vitro environments can consist of, but are not limited to, test tubes and cell culture.

The term “in vivo” refers to the natural environment (e.g., an animal or a cell) and to processes or reaction that occur within a natural environment.

The terms “peptide”, “polypeptide” and “protein” are used interchangeably herein to refer to polymers of amino acids (e.g., naturally occurring amino acids and non-natural amino acids) of any length. The terms also encompass an amino acid polymer that has been modified; for example, disulfide bond formation, glycosylation, acetylation, phosphorylation, lipidation, or conjugation with a labeling component.

By “pharmaceutical composition” is meant any composition (e.g., gel-based inulin formulations associated with one or more allergens) (e.g., a gel-based inulin formulation associated with one or more allergens and one or both of a prebiotic compound and a therapeutic agent) that is suitable for administration to a subject.

By “pharmaceutically acceptable diluent, excipient, carrier, or adjuvant” is meant a diluent, excipient, carrier, or adjuvant which is physiologically acceptable to the subject while retaining the therapeutic properties of the pharmaceutical composition with which it is administered.

As used herein, the term “sample” is used in its broadest sense. In one sense, it is meant to include a specimen or culture obtained from any source, as well as biological and environmental samples. Biological samples may be obtained from animals (including humans) and encompass fluids, solids, tissues, and gases. Biological samples include blood products, such as plasma, serum and the like. Environmental samples include environmental material such as surface matter, soil, water, crystals, and industrial samples. Such examples are not however to be construed as limiting the sample types applicable to the present invention.

As used herein, the term “subject” refers to any animal (e.g., a mammal), including, but not limited to, humans, non-human primates, rodents, and the like, which is to be the recipient of a particular treatment. Typically, the terms “subject” and “patient” are used interchangeably herein in reference to a human subject.

By “therapeutically effective amount” is meant the amount of a composition (e.g., gel-based inulin formulations associated with one or more allergens) (e.g., a gel-based inulin formulation associated with one or more allergens and one or both of a prebiotic compound and a therapeutic agent) administered to improve, inhibit, or ameliorate a condition of a subject, or a symptom of a disorder or disease, e.g., an allergy, in a clinically relevant manner. Any improvement in the subject is considered sufficient to achieve treatment. Preferably, an amount sufficient to treat is an amount that reduces, inhibits, or prevents the occurrence or one or more symptoms of the disease or disorder or is an amount that reduces the severity of, or the length of time during which a subject suffers from one or more symptoms of the disease or disorder (e.g., by at least about 10%, about 20%, or about 30%, more preferably by at least about 50%, about 60%, or about 70%, and most preferably by at least about 80%, about 90%, about 95%, about 99%, or more, relative to a control subject that is not treated with a composition described herein). An effective amount of the pharmaceutical composition used to practice the methods described herein (e.g., the treatment, prevention, and/or amelioration of allergies (e.g., food allergies)) varies depending upon the manner of administration and the age, body weight, and general health of the subject being treated. A physician or researcher can decide the appropriate amount and dosage regimen.

### DETAILED DESCRIPTION

Allergy is a disorder of the immune system and is characterized by the occurrence of allergic reactions to normally harmless environmental substances. Allergies are caused by allergens, which may be present in a wide variety of sources, including but not limited to pollens or other plant components, dust, molds or fungi, foods, additives, latex, transfusion reactions, animal or bird danders, insect venoms, radiocontrast medium, medications or chemicals. Common allergic reactions include eczema, hives, hay fever, asthma, and reactions to venoms. Mild allergies like hay fever are highly prevalent in the human population and cause symptoms such as allergic conjunctivitis, itchiness, and runny nose. In some people, severe allergies to environmental or dietary allergens or to medication may result in life-threatening anaphylactic reactions and potentially death, if left untreated.

Allergic reactions can occur in three distinct patterns: a) an early phase reaction or acute response, b) late phase reactions and c) potentially chronic allergic inflammation. The early phase of the allergic reaction typically occurs within minutes, or even seconds, following a first allergen exposure, where this early phase is also commonly referred to as the immediate allergic reaction. In the early stages of allergy, a hypersensitivity reaction against an allergen, encountered for the first time, causes a response in Th2 cells, which are a subset of T cells that produce the cytokine interleukin-4 (IL-4). The Th2 cells interact with B cells (lymphocytes that produce antibodies against antigens) and, coupled with the effects of IL-4, stimulate the B cells to begin production and secretion of Immunoglobulin E (IgE).

IgE plays an important role in allergies and allergic reactions. Upon introduction of an allergen, B cells of the respective subject produce large amounts of IgE. The IgE elicits an immune response by binding onto receptors found on basophils and mast cells. When activated, these cells release chemical mediators such as histamine and cytokines that cause the characteristic symptoms of allergy.

A food allergy is an adverse immune response to a food allergen, e.g., a food protein. Common food allergens are found in shellfish, peanuts, tree nuts, fish, milk, eggs, soy and fresh fruits such as strawberries, mango, banana, and apple Immunoglobulin E (IgE)-mediated food allergies are classified as type-I immediate hypersensitivity reactions. These allergic reactions have an acute onset (from seconds to one hour) and the accompanying symptoms may include angioedema (soft tissue swelling of the eyelids, face, lips, tongue, larynx and trachea); hives; itching of the mouth, throat, eyes, or skin; gastrointestinal symptoms such as nausea, vomiting, diarrhea, stomach cramps, or abdominal pain; rhinorrhea or nasal congestion; wheezing, shortness of breath, or difficulty swallowing; and even anaphylaxis, a severe, whole-body allergic reaction that can result in death. 1 out of 12 children under the age of 21 years of age have a doctor’s diagnosis of food allergies (see, Gupta, et al., JAMA Pediatrics, November 2013, Vol. 167, No. 11). This epidemic has been reported to be doubling every 10 years for certain nuts (CDC 2009). Moreover, there are still deaths that occur every year due fatal food allergies. Importantly, over $24 billion is spent per year on health care/care costs for food allergic reactions (see, Gupta, et al., JAMA Pediatrics, November 2013, Vol. 167, No. 11). This cost is largely due to about 90,000 visits to the ER per year in the U.S. due to food induced anaphylactic symptoms.

According to the World Health Organization statistics on allergy, the incidence of allergy has been on the rise in industrialized countries over the past 50 years, and nearly 40-50% of school-aged children world-wide being sensitive to at least one common allergen (see, Pawankar R, et al. The WAO White Book on Allergy (Update 2013)). Although allergy may arise during childhood, it is also possible for allergies to develop or arise throughout one’s life.

The severity of an allergic reaction upon exposure to an allergen can range broadly from mild symptoms to sometimes fatal reactions. The symptoms and severity of an allergy may depend on factors such as type of immune response(s) involved, the duration and magnitude of the immune response(s), amount of allergen, and the site of contact/exposure to the allergen. Examples of allergy symptoms include, without limitation, skin rash, skin redness, hives, skin bumps/patches/welts, itchy/watery eyes, headache, sneezing, wheezing, shortness of breath, chest tightness, cough, runny nose, sore throat, swelling, nausea, vomiting, diarrhea, and anaphylaxis.

A subject may contact or be exposed to an allergen that induces an allergic reaction by any route known in the art, for example, through ingestion, inhalation, injection, or direct contact. The symptoms associated with the allergic reaction may be localized to the site of contact or exposure to the allergen, for example a region of the skin, respiratory tract, or gastrointestinal tract, a distal site, or may become systemic, such as in the case of anaphylaxis.

Immune responses stimulated in response to contact or exposure to an allergen may be referred to as allergic reactions. In general, an allergic reaction may occur immediately after contact or exposure to an allergen or within about a half-hour or longer after contact or exposure.

Experiments conducted during the course of developing embodiments for the present invention resulted in the development of a platform technology for oral immunotherapy against allergies (e.g., food allergies (e.g., peanut allergy). In particular, such experiments resulted in the development of a new oral fiber-gel formulation based on dietary natural fiber that people are widely consuming. Indeed, inulin was formulated into a new oral inulin-gel, and an oral inulin-gel containing peanut extract was developed. When administered orally, results demonstrated that inulin-gel containing peanut extract effectively protected mice against peanut allergen challenge as demonstrated by no sign of anaphylactic shock, bodyweight loss, or body temperature drop. Such results indicate that inulin-gel modulates the gut microbiome to promote the induction of regulatory T cells (e.g., a subset of CD4 T cells known to be crucial for immune tolerance for food allergens and other autoantigens).

Accordingly, the present invention relates generally to compositions comprising a gel-based inulin formulation associated with (e.g., complexed, conjugated, encapsulated, absorbed, adsorbed, admixed) one or more therapeutic agents (e.g., immunomodulatory agent, immunosuppressant, allergen) and related methods for the treatment of autoimmune disorders (e.g., colitis) (e.g, allergy, such as food allergy). Also provided herein are compositions and methods for modulating an immune response associated with an autoimmune disorder (e.g., allergy) and/or inducing immune tolerance or desensitization to an autoimmune disorder (e.g., allergy, such as a food allergy).

In certain embodiments, the present invention provides a composition comprising a gel-based inulin formulation associated with (e.g., complexed, conjugated, encapsulated, absorbed, adsorbed, admixed) one or more therapeutic agents (e.g., immunomodulatory agent, immunosuppressant, allergen). In some embodiments, the gel-based inulin formulation has an average degree of polymerization at or higher than 20 and at or less than 47. In some embodiments, the gel-based inulin formulation has an average degree of polymerization at approximately 26 (e.g., 20, 21, 22, 23, 24, 25, 26, 27, 28, 29, 30, 31, 32).

Inulin is a polysaccharide belonging to the fructan group. It consists of a beta-2-1-linked chain of fructose molecules, this chain having at its end an alpha-D-glucose unit at the reducing end. Inulin occurs in economically recoverable amounts in various plants such as, for example, chicory roots and dahlia tubers. In addition, inulin has been found for example in Jerusalem artichokes and artichokes. The average chain lengths of the various inulins and their physicochemical properties differ from plant species to plant species.

In some embodiments, the gel-based inulin formulation further comprises one or more prebiotic compounds selected from a fructo-oligosaccharide, a short-chain fructo-oligosaccharide, an isomalt-oligosaccharide, a transgalacto-oligosaccharide, a pectin, a xylo-oligosaccharide, a chitosan-oligosaccharide, a beta-glucan, an arable gum modified starch, a resistant potato starch, guar gum, bean gum, gelatin, glycerol, a polydextrose, a D-tagatose, an acacia fiber, carob, an oat, and a citrus fiber.

In some embodiments, the therapeutic agent is an immunomodulatory agent selected from fingolimod; 2-(1′H-indole-3′-carbonyl)-thiazole-4-carboxylic acid methyl ester (ITE) or related ligands; Trichostatin A; Suberoylanilide hydroxamic acid (SAHA); statins; mTOR inhibitors; TGF-β signaling agents; TGF-β receptor agonists; histone deacetylase inhibitors; corticosteroids; inhibitors of mitochondrial function; NF-κβ inhibitors; adenosine receptor agonists; prostaglandin E2 agonists (PGE2; phosphodiesterase inhibitors; proteasome inhibitors; kinase inhibitors; G-protein coupled receptor agonists; G-protein coupled receptor antagonists; glucocorticoids; retinoids; cytokine inhibitors; cytokine receptor inhibitors; cytokine receptor activators; peroxisome proliferator-activated receptor antagonists; peroxisome proliferator-activated receptor agonists; histone deacetylase inhibitors; calcineurin inhibitors; phosphatase inhibitors; PI3 KB inhibitors; autophagy inhibitors; aryl hydrocarbon receptor inhibitors; proteasome inhibitor I (PSI); oxidized ATPs IDO; vitamin D3; cyclosporins; aryl hydrocarbon receptor inhibitors; resveratrol; azathiopurine (Aza); 6-mercaptopurine (6-MP); 6-thioguanine (6-TG); FK506; sanglifehrin A; salmeterol; mycophenolate mofetil (MMF); aspirin and other COX inhibitors; niflumic acid; estriol; triptolide; OPN-305, OPN-401; Eritoran (E5564); TAK-242; Cpn10; NI-0101; 1A6; AV411; IRS-954 (DV-1079); IMO-3100; CPG-52363; CPG-52364; OPN-305; ATNC05; NI-0101; IMO-8400; Hydroxychloroquine; CU-CPT22; C29; Ortho-vanillin; SSL3 protein; OPN-305; 5 SsnB; Vizantin; (+)-N-phenethylnoroxymorphone; VB3323; Monosaccharide 3; (+)-Naltrexone and (+)-naloxone; HT52; HTB2; Compound 4a; CNTO2424; TH1020; INH-ODN; E6446; AT791; CpG ODN 2088; ODN TTAGGG; COV08-0064; 2R9; GpG oligonucleotides; 2-aminopurine; Amlexanox; Bay 1 1-7082; BX795; CH-223191; Chloroquine; CLI-095; CU-CPT9a; Cyclosporin A; CTY387; Gefitnib; Glybenclamide; H-89; H-131; Isoliquiritigenin; MCC950; MRT67307; OxPAPC; Parthenolide; Pepinh-MYD; Pepinh-TRIF; Polymyxin B; R406; RU.521; VX-765; YM201636; Z-VAD-FMK; and AHR-specific ligands; including but not limited to 2,3,7,8-tetrachloro-dibenzo-p-dioxin (TCDD); tryptamine (TA); and 6 formylindolo[3,2 b]carbazole (FICZ))).

In particular embodiments, the therapeutic agent is an immunosuppressant selected from fingolimod; 2-(1′H-indole-3′-carbonyl)-thiazole-4-carboxylic acid methyl ester (ITE) or related ligands; Trichostatin A; and/or Suberoylanilide hydroxamic acid (SAHA). In some embodiments, the at least one therapeutic agent is included within the gel-based inulin formulation. In some embodiments, the gel-based inulin formulation is further admixed with an adjuvant (e.g., CPG, polyIC, poly-ICLC, 1018 ISS, aluminum salts, Amplivax, AS15, BCG, CP-870,893, CpG7909, CyaA, dSLIM, GM-CSF, IC30, IC31, Imiquimod, ImuFact IMP321, IS Patch, ISS, ISCOMATRIX, Juvlmmune, LipoVac, MF59, monophosphoryl lipid A, Montanide IMS 1312, Montanide ISA 206, Montanide ISA 50V, Montanide ISA-51, OK-432, OM-174, OM-197-MP-EC, ONTAK, PepTel.RTM, vector system, PLGA microparticles, imiquimod, resiquimod, gardiquimod, 3M-052, SRL172, Virosomes and other Virus-like particles, YF-17D, VEGF trap, beta-glucan, Pam3Cys, Aquila’s QS21 stimulon, vadimezan, AsA404 (DMXAA), and any derivative of an adjuvant).

In some embodiments, the composition does not contain an adjuvant.

Such compositions wherein the therapeutic agent is an allergen, upon administration in a therapeutically effective amount to a subject (e.g., a human subject) are capable of one or more treating an allergy (e.g., a food allergy), reducing one or more symptom associated with an allergy (e.g., food allergy), modulating one or more immune responses associated with an allergy (e.g., food allergy), inducing the proliferation and/or accumulation of regulatory T cells in the subject, suppressing the production of IgE antibodies (e.g., total IgE antibodies or allergen-specific IgE antibodies), suppressing one or more Th2 immune responses, allowing the subject to survive a challenge with the allergen (e.g., in case of an anaphylactic allergic response in the inadvertent exposure to a peanut allergen).

Such compositions are not limited to a particular type or kind of allergen. Sources of allergens include animal products, plants, food, insect stings, drugs, fungal spores, and microorganisms.

Examples of allergens from animal products include fur, dander, cockroach calyx, wool, dust mite excretion, and fel d 1 (e.g., a protein produced in cat saliva and sebaceous glands). Examples of allergens from plant include plant pollens from grass such as ryegrass; weeds such as ragweed, nettle, sorrel; and trees such as birch, alder, hazel, oak, elm, and maple. Also, urushiol is a resin produced by poison ivy and poison oak that is an allergen which causes skin rash. Examples of food allergens include peanuts, tree nuts such as pecans and almonds, eggs, milk, shellfish, fish, wheat and their derivative, soy and their derivatives. Examples of allergens from insect stings include bee sting, wasp sting, and mosquito stings. Examples of drug allergens include penicillin, sulfonamides, quinidine, phenylbutazone, thiouracils, methyldopa, hydantoins, and salicytates. Example of fungal allergens include basidiospores such as Ganoderma; mushroom spores; allergens from the aspergillus and alternaria-penicillin families; and cladosporium spores. Examples of microorganisms that can cause an allergic reaction include viruses and bacteria. Other allergens include latex, metal, wood, chemicals, cosmetics, dyes, vaccines, hormones, vegetables, fruits, sugars, animals and essentially anything under the sun, including the sun itself. Another example of an allergen can be semen. Infertility can be caused by sensitization of a woman to her partner’s semen. This is a true allergy that may prevent conception in the normal way leading to increased medical expenses.

In some embodiments, two or more allergens are associated with (e.g., complexed, conjugated, encapsulated, absorbed, adsorbed, admixed) with the gel-based inulin formulation. In some embodiments, three or more allergens are associated with (e.g., complexed, conjugated, encapsulated, absorbed, adsorbed, admixed) with the gel-based inulin formulation. In some embodiments, four or more allergens are associated with (e.g., complexed, conjugated, encapsulated, absorbed, adsorbed, admixed) with the gel-based inulin formulation. In some embodiments, five or more allergens are associated with (e.g., complexed, conjugated, encapsulated, absorbed, adsorbed, admixed) with the gel-based inulin formulation. In some embodiments, six or more allergens are associated with (e.g., complexed, conjugated, encapsulated, absorbed, adsorbed, admixed) with the gel-based inulin formulation. In some embodiments, seven or more allergens are associated with (e.g., complexed, conjugated, encapsulated, absorbed, adsorbed, admixed) with the gel-based inulin formulation. In some embodiments, eight or more allergens are associated with (e.g., complexed, conjugated, encapsulated, absorbed, adsorbed, admixed) with the gel-based inulin formulation. In some embodiments, nine or more allergens are associated with (e.g., complexed, conjugated, encapsulated, absorbed, adsorbed, admixed) with the gel-based inulin formulation. In some embodiments, ten or more (e.g., 10, 11, 12, 15, 20, 25, 50, 75, 100, 1000, 10,000, etc.) allergens are associated with (e.g., complexed, conjugated, encapsulated, absorbed, adsorbed, admixed) with the gel-based inulin formulation. In such embodiments, the allergens associated with (e.g., complexed, conjugated, encapsulated, absorbed, adsorbed, admixed) with the gel-based inulin formulation can be identical allergens or different allergens. In such embodiments wherein the allergens are different, the gel-based inulin formulation can accommodate any desired ratio or proportion of the different kinds of allergens within the composition.

Such compositions are configured for any desired manner of administration to a subject. In some embodiments, the agent is administered orally (e.g., by oral gavage). In some embodiments, the agent is administered via acupuncture. In some embodiments, administration can be by any suitable route of administration including buccal, dental, endocervical, intramuscular, inhalation, intracranial, intralymphatic, intramuscular, intraocular, intraperitoneal, intrapleural, intrathecal, intratracheal, intrauterine, intravascular, intravenous, intravesical, intranasal, ophthalmic, otic, biliary perfusion, cardiac perfusion, priodontal, rectal, spinal subcutaneous, sublingual, topical, intravaginal, transermal, ureteral, or urethral.

Examples of autoimmune disorders that can be treated according to the compositions (e.g., a composition comprising a gel-based inulin formulation associated with one or more therapeutic agents (e.g., immunomodulatory agent, immunosuppressant, allergen)) (e.g., a composition comprising a gel-based inulin formulation associated with one or more therapeutic agents (e.g., immunomodulatory agent, immunosuppressant, allergen) and a prebiotic compound) and methods provided herein, include without limitation, allergic asthma, allergic colitis, animal allergies, atopic allergies, hay fever, skin allergy, hives, atopic dermatitis, anaphylaxis, allergic rhinitis, drug or medicinal allergy, eczema (atopic dermatitis), food allergy, fungal allergy, insect allergy (including insect bite/venom allergies), mold allergies, plant allergies, and pollenosis. In some embodiments, the allergy is a food allergy.

Aspects of the present disclosure relate to treating food allergy and/or modulating an immune response associated with a food allergy in a subject. Also provided herein are methods of inducing immune tolerance or desensitization to a food allergy. As used herein, the term “food allergy” refers to an undesired allergic immune response to a food, or specifically, to an allergen present in the food. In some embodiments, an allergic reaction associated with a food allergy is induced following contact, for example through ingestion, of a food or foods containing the same or similar allergens. As will be evident to one of skill in the art, the symptoms associated with the food allergy may manifest in the gastrointestinal tract of the subject, for example, following ingestion with food containing the allergen; however, the allergic reaction may affect other sites, such as the respiratory tract or skin.

Food allergies are generally considered to be IgE-mediated immune reactions, however non-IgE-mediated food allergies as well as mixed IgE-mediated/non-IgE-mediated food allergies exist. IgE-mediated food allergies tend to occur immediately or within about 2 hours following contact with the allergen and include hives (acute uticaria), angioedema, swelling, anaphylaxis, food-associated exercise-induced anaphylaxis, oral allergy syndrome, and/or immediate gastrointestinal hypersensitivity involving vomiting and pain. Non-IgE-mediated immune responses involved in food allergy, also referred to as cell-mediated responses, are delayed hypersensitivity reactions and may involve food protein-induced enterocolitis syndrome, food protein-induced allergic proctocolitis, allergic contact dermatitis, and Heiner syndrome. Mixed or combined IgE-mediated/non-IgE-mediated immune responses involved in food allergy are associated with both IgE and T cell mediated effects and may include atopic dermatitis, eosinophilic esophagitis, and/or eosinophilic gastroenteritis.

In some embodiments, the compositions and methods described herein (e.g., a composition comprising a gel-based inulin formulation associated with one or more therapeutic agents (e.g., immunomodulatory agent, immunosuppressant, allergen)) (e.g., a composition comprising a gel-based inulin formulation associated with one or more therapeutic agents (e.g., immunomodulatory agent, immunosuppressant, allergen) and a prebiotic compound) are used to treat an IgE-mediated food allergy. In some embodiments, the compositions and methods described herein are used to modulate an immune response associated with an IgE-mediated food allergy. In some embodiments, the compositions and methods described herein are used to induce immune tolerance or desensitization to an IgE-mediated food allergy. The compositions and methods described herein may also be used in the context of non-IgE mediated food allergies and/or mixed or combined IgE-mediated/non-IgE-mediated food allergies.

Examples of food allergies include, without limitation, peanut allergy, tree nut allergy, egg allergy, corn allergy, fruit allergy, milk allergy, garlic allergy, soy allergy, wheat allergy, seafood allergy, fish allergy (e.g., shellfish allergy), and seed allergy (e.g., sesame seed allergy).

Non-limiting examples of foods containing allergens to which a food allergy may occur include abalone (perlemoen), acerola, Alaska pollock, almond, aniseed, apple, apricot, avocado, banana, barley, bell pepper, Brazil nut, buckwheat, cabbage, carp, carrot, cashew, caster bean, celery, celeriac, cherry, chestnut, chickpea (garbanzo, bengal gram), cococa, coconut, cod, cotton seed, courgett (zucchini), crab, date, egg, fig, fish, flax seed (linseed), frog, garden plum, garlic, grape, hazelnut, kiwi fruit (Chinese gooseberry), lentil, lettuce, lobster, lupin (lupine), lychee, mackerel, maize (corn), mango, melon, milk, mustard, oat oyster, peach, peanut (ground nuts, monkey nuts), pear, pecan, persimmon, pine nut, pineapple, pomegranate, poppy seed, potato, pumpkin, rice, rye, salmon, sesame, sesame seed, shrimp (black tiger shrimp, brown shrimp, greasyback shrimp, Indian prawn, Neptune rose shrimp, white shrimp), snail, soybean (soya), squid, strawberry, sunflower seed, tomato, tuna, turnip, walnut, and wheat (bread-making wheat, pasta wheat, kamut, spelt).

In one aspect, the disclosure provides compositions (e.g., a composition comprising a gel-based inulin formulation associated with one or more therapeutic agents (e.g., immunomodulatory agent, immunosuppressant, allergen)) (e.g., a composition comprising a gel-based inulin formulation associated with one or more therapeutic agents (e.g., immunomodulatory agent, immunosuppressant, allergen) and a prebiotic compound) and methods of treatment for a disease or disorder, such as allergy (e.g., food allergy), in a subject. As used herein, “subject,” “individual,” and “patient” are used interchangeably, and refer to a vertebrate, preferably a mammal such as a human. Mammals include, but are not limited to, human primates, non-human primates or murine, bovine, equine, canine or feline species. In some embodiments, the subject is a human. In some embodiments, the human subject is a neonatal subject, a pediatric subject, an adolescent subject, an adult subject, or a geriatric subject. In some embodiments, the subject has or is at risk of having an allergy, such as a food allergy. In some embodiments, the subject has had one or allergic reactions following contact or exposure to a particular food or group of foods containing an allergen. In some embodiments, the subject has had a medical history associated with allergy, such as a food allergy. In some embodiments, the subject has a family history of allergy or of an allergy to a specific allergen. For example, a family history may influence the likelihood for that subject to have or develop an allergy, such as a food allergy. Additionally, a subject having a food allergy to a specific food (e.g., specific allergen in a food) may also predispose that subject to have or develop a food allergy to a different food (e.g., a different specific allergen in a food).

In some embodiments, the subject has a risk factor associated with developing an allergy. Examples of risk factors associated with the development of a food allergy include, without limitation, an immature mucosal immune system, early introduction of solid food, hereditary increase in mucosal permeability, IgA deficiency or delayed IgA production, inadequate challenge of the intestinal immune system with commensal flora, genetically determined bias toward Th2 immune responses, polymorphisms of Th2 cytokine or IgE receptor genes, impaired enteric nervous system, immune alterations (e.g., low levels of TGF-β).

Any of the compositions described herein (e.g., gel-based inulin formulations associated with one or more therapeutic agents (e.g., immunomodulatory agent, immunosuppressant, allergen)) (e.g., a gel-based inulin formulation associated with one or more therapeutic agents (e.g., immunomodulatory agent, immunosuppressant, allergen) and a prebiotic compound) may be administered to a subject in a therapeutically effective amount or a dose of a therapeutically effective amount to treat or prevent a disease or disorder (e.g., food allergy). The terms “treat” or “treatment” refer to reducing or alleviating one or more of the symptoms associated with a disease (e.g., an allergy such as food allergy). The terms “prevent” or “prevention” encompass prophylactic administration and may reduce the incidence or likelihood of the occurrence of the disease or disorder (e.g., food allergy). In some embodiments, the composition reduces the incidence or likelihood of the occurrence of an allergic reaction, such as an allergic reaction associated with a food or food allergen. For instance, in some embodiments, administration of the compositions provided herein result in an altered microbiome in the subject that provides an effect in a subject that reduces the incidence or likelihood of an allergic reaction. For instance, in some embodiments, administration of the compositions provided herein result in a healthy microbiome in the subject that provides an effect in a subject that reduces the incidence or likelihood of an allergic reaction. In some embodiments, administration of the composition provided herein result in a reduction or alleviation of one or more symptom associated with allergy, such as a symptom associated with an allergic reaction.

In some embodiments, the compositions and methods described herein (e.g., gel-based inulin formulations associated with one or more therapeutic agents (e.g., immunomodulatory agent, immunosuppressant, allergen)) (e.g., a gel-based inulin formulation associated with one or more therapeutic agents (e.g., immunomodulatory agent, immunosuppressant, allergen) and a prebiotic compound) are used to induce immune tolerance to an allergen associated with an allergy (e.g., a food allergy) or desensitize an immune response to an allergen associated with an allergy (e.g., a food allergy). As used herein, the terms “tolerance” and “immune tolerance” in the context of allergy refer to a reduced responsiveness or non-responsiveness of the immune response to one or more stimuli, such as an allergen associated with allergy. In particular, tolerance or immune tolerance refer to reduced responsiveness or non-responsiveness of the immune response to one or more stimuli over a sustained or long term period of time. In contrast, the term “desensitize” in the context of allergy refers a reversible state of reduced responsiveness or non-responsiveness of the immune response to one or more stimuli, for example during the course of a desensitization regimen.

In some embodiments, the compositions and methods described herein (e.g., gel-based inulin formulations associated with one or more therapeutic agents (e.g., immunomodulatory agent, immunosuppressant, allergen)) (e.g., a gel-based inulin formulation associated with one or more therapeutic agents (e.g., immunomodulatory agent, immunosuppressant, allergen) and a prebiotic compound) used to modulate an immune response associated with an allergy (e.g., a food allergy). As will be evident to one of skill in the art, the compositions and methods described herein may enhance one or more immune response(s) associated with an allergy and reduce or suppress one or more other immune response(s) associated with the allergy.

In some embodiments, the compositions and methods described herein (e.g., gel-based inulin formulations associated with one or more therapeutic agents (e.g., immunomodulatory agent, immunosuppressant, allergen)) (e.g., a gel-based inulin formulation associated with one or more therapeutic agents (e.g., immunomodulatory agent, immunosuppressant, allergen) and a prebiotic compound) induce the proliferation and/or accumulation of regulatory T cells. Regulatory T cells can generally be characterized by the expression of FoxP3, CD25, and CD4. In some embodiments, administration of the compositions described herein results in an increase in the proliferation and/or accumulation of regulatory T cells in the subject (e.g., total regulatory T cells or allergen-specific regulatory T cells) by at least 1.1-fold, 1.2-fold, 1.3-fold, 1.4-fold, 1.5-fold, 2-fold, 3-fold, 4-fold, 5-fold, 6-fold, 7-fold, 8-fold, 9-fold, 10-fold, 20-fold, 30-fold, 40-fold, 50-fold, 100-fold, 1000-fold, 10⁴-fold, 10⁵-fold or more, as compared to the quantity of regulatory T cells in the subject prior to administration of the compositions. In some embodiments, administration of the compositions described herein results in an increase the proliferation and/or accumulation of regulatory T cells (e.g., total regulatory T cells or allergen-specific regulatory T cells) by at least 1.5-fold, 2-fold, 3-fold, 4-fold, 5-fold, 6-fold, 7-fold, 8-fold, 9-fold, 10-fold, 20-fold, 30-fold, 40-fold, 50-fold, 100-fold, 1000-fold, 10⁴-fold, 10⁵-fold or more, as compared to the quantity of regulatory T cells in another subject (e.g., a reference subject) who did not receive the compositions.

In some embodiments, administration of the compositions described herein (e.g., gel-based inulin formulations associated with one or more therapeutic agents (e.g., immunomodulatory agent, immunosuppressant, allergen)) (e.g., a gel-based inulin formulation associated with one or more therapeutic agents (e.g., immunomodulatory agent, immunosuppressant, allergen) and a prebiotic compound) results in an increase the proliferation and/or accumulation of regulatory T cells (e.g., total regulatory T cells or allergen-specific regulatory T cells) by at least 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 35%, 40%, 45%, 50%, 55%, 60%, 65%, 70%, 75%, 80%, 85%, 90%, 95%, 100%, 125%, 150% or more, as compared to the quantity of regulatory T cells in the subject (or particular site in the subject) prior to administration of the compositions. In some embodiments, administration of the compositions described herein results in an increase the proliferation and/or accumulation of regulatory T cells (e.g., total regulatory T cells or allergen-specific regulatory T cells) by at least 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 35%, 40%, 45%, 50%, 55%, 60%, 65%, 70%, 75%, 80%, 85%, 90%, 95%, 100%, 125%, 150% or more, as compared to the quantity of regulatory T cells in another subject (e.g., a reference subject) who did not receive the compositions.

The induction of regulatory T cells and corresponding allergy treatment are intricately related. In some embodiments, in the treatment of one or more allergies, it is desired to have a regulatory T cells induction that is a range associated with treatment efficacy for the one more allergies. In some embodiments, for a particular allergy treatment regimen it is desired to have a regulatory T cells response that is significantly strong to induce the desired allergy treatment effect, but not so strong as to result in undesired immunological events. In some embodiments, administration of the compositions described herein results in an increase the proliferation and/or accumulation of regulatory T cells (e.g., total regulatory T cells or allergen-specific regulatory T cells) by between 1% and 20%, 2% and 19%, 3% and 17%, 4% and 16%, 4% and 15%, 5% and 15%, 6% and 14%, 7% and 13%, 8% and 12%, 5% and 10%, 5% and 15%, 10% and 15%, or 8% and 15% as compared to the quantity of regulatory T cells in the subject (or particular site in the subject) prior to administration of the compositions. In some embodiments, administration of the compositions described herein results in an increase the proliferation and/or accumulation of regulatory T cells (e.g., total regulatory T cells or allergen-specific regulatory T cells) by between 1% and 20%, 2% and 19%, 3% and 17%, 4% and 16%, 4% and 15%, 5% and 15%, 6% and 14%, 7% and 13%, 8% and 12%, 5% and 10%, 5% and 15%, 10% and 15%, or 8% and 15% as compared to the quantity of regulatory T cells in another subject (e.g., a reference subject) who did not receive the compositions.

In some embodiments, administration of the compositions described herein (e.g., gel-based inulin formulations associated with one or more therapeutic agents (e.g., immunomodulatory agent, immunosuppressant, allergen)) (e.g., a gel-based inulin formulation associated with one or more therapeutic agents (e.g., immunomodulatory agent, immunosuppressant, allergen) and a prebiotic compound) results in an increase in activity of regulatory T cells (e.g., total regulatory T cells or allergen-specific regulatory T cells) at a particular site in the subject. In some embodiments, administration of the compositions described herein results in an increase in activity of regulatory T cells (e.g., total regulatory T cells or allergen-specific regulatory T cells) by at least 1.1-fold, 1.2-fold, 1.3-fold, 1.4-fold, 1.5-fold, 2-fold, 3-fold, 4-fold, 5-fold, 6-fold, 7-fold, 8-fold, 9-fold, 10-fold, 20-fold, 30-fold, 40-fold, 50-fold, 100-fold, 1000-fold, 10⁴-fold, 10⁵-fold or more, as compared to the activity of regulatory T cells in the subject prior to administration of the compositions. In some embodiments, administration of the compositions described herein results in an increase in activity of regulatory T cells (e.g., total regulatory T cells or allergen-specific regulatory T cells) by at least 1.5-fold, 2-fold, 3-fold, 4-fold, 5-fold, 6-fold, 7-fold, 8-fold, 9-fold, 10-fold, 20-fold, 30-fold, 40-fold, 50-fold, 100-fold, 1000-fold, 10⁴-fold, 10⁵-fold or more, as compared to the activity of regulatory T cells in another subject (e.g., a reference subject) who did not receive the compositions.

In some embodiments, administration of the compositions described herein (e.g., gel-based inulin formulations associated with one or more therapeutic agents (e.g., immunomodulatory agent, immunosuppressant, allergen)) (e.g., a gel-based inulin formulation associated with one or more therapeutic agents (e.g., immunomodulatory agent, immunosuppressant, allergen) and a prebiotic compound) results in an increase in the activity of regulatory T cells (e.g., total regulatory T cells or allergen-specific regulatory T cells) by at least 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 35%, 40%, 45%, 50%, 55%, 60%, 65%, 70%, 75%, 80%, 85%, 90%, 95%, 100%, 125%, 150% or more, as compared to the activity of regulatory T cells in the subject prior to administration of the compositions. In some embodiments, administration of the compositions described herein results in an increase in the activity of regulatory T cells (e.g., total regulatory T cells or allergen-specific regulatory T cells) by at least 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 35%, 40%, 45%, 50%, 55%, 60%, 65%, 70%, 75%, 80%, 85%, 90%, 95%, 100%, 125%, 150% or more, as compared to the activity of regulatory T cells in another subject (e.g., a reference subject) who did not receive the compositions.

The abundance of regulatory T cells (e.g., total regulatory T cells or allergen-specific regulatory T cells) can be assessed by any method known in the art, for example by detecting a cellular marker indicative of regulatory T cells (e.g., FoxP3), assessing a direct or indirect activity of regulatory T cells, and/or by measuring the production of one or more cytokines produced by regulatory T cells (e.g., IL-10).

In some embodiments, the compositions and methods described herein (e.g., gel-based inulin formulations associated with one or more therapeutic agents (e.g., immunomodulatory agent, immunosuppressant, allergen)) (e.g., a gel-based inulin formulation associated with one or more therapeutic agents (e.g., immunomodulatory agent, immunosuppressant, allergen) and a prebiotic compound) suppress the production of IgE antibodies. In some embodiments, the compositions and methods suppress the production of total IgE antibodies in the subject. In some embodiments, the compositions and methods suppress the production of IgE antibodies that are specific to an allergen (e.g., allergen-specific IgE antibodies) associated with an allergy, e.g., a food allergen associated with a food allergy. In some embodiments, administration of the compositions described herein results in levels of IgE antibodies (e.g., total IgE antibodies or allergen-specific IgE antibodies) that are reduced by at least 1.5-fold, 2-fold, 3-fold, 4-fold, 5-fold, 6-fold, 7-fold, 8-fold, 9-fold, 10-fold, 20-fold, 30-fold, 40-fold, 50-fold, 100-fold, 1000-fold, 10⁴-fold, 10⁵-fold or more, as compared to the level of IgE antibodies (e.g., total IgE antibodies or allergen-specific IgE antibodies) in the subject (or a sample thereof) prior to administration of the compositions. In some embodiments, administration of the compositions described herein results in levels of IgE antibodies (e.g., total IgE antibodies or allergen-specific IgE antibodies) that are reduced by at least 1.5-fold, 2-fold, 3-fold, 4-fold, 5-fold, 6-fold, 7-fold, 8-fold, 9-fold, 10-fold, 20-fold, 30-fold, 40-fold, 50-fold, 100-fold, 1000-fold, 10⁴-fold, 10⁵-fold or more, as compared to the level of IgE antibodies in another subject (e.g., a reference subject) who did not receive the compositions.

In some embodiments, administration of the compositions described herein (e.g., gel-based inulin formulations associated with one or more therapeutic agents (e.g., immunomodulatory agent, immunosuppressant, allergen)) (e.g., a gel-based inulin formulation associated with one or more therapeutic agents (e.g., immunomodulatory agent, immunosuppressant, allergen) and a prebiotic compound) results in levels of IgE antibodies (e.g., total IgE antibodies or allergen-specific IgE antibodies) that are reduced by at least 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 35%, 40%, 45%, 50%, 55%, 60%, 65%, 70%, 75%, 80%, 85%, 90%, 95%, or 100% as compared to the level of IgE antibodies (e.g., total IgE antibodies or allergen-specific IgE antibodies) in the subject (or a sample thereof) prior to administration of the compositions. In some embodiments, administration of the compositions described herein results in levels of IgE antibodies (e.g., total IgE antibodies or allergen-specific IgE antibodies) that are reduced by at least 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 35%, 40%, 45%, 50%, 55%, 60%, 65%, 70%, 75%, 80%, 85%, 90%, 95%, or 100% as compared to the level of IgE antibodies in another subject (e.g., a reference subject) who did not receive the compositions.

In some embodiments, administration of the compositions described herein (e.g., gel-based inulin formulations associated with one or more therapeutic agents (e.g., immunomodulatory agent, immunosuppressant, allergen)) (e.g., a gel-based inulin formulation associated with one or more therapeutic agents (e.g., immunomodulatory agent, immunosuppressant, allergen) and a prebiotic compound) results in levels of IgE antibodies (e.g., total IgE antibodies or allergen-specific IgE antibodies) that are reduced by between 30% and 50%, 30% and 45%, 35% and 45%, 30% and 40%, 35% and 40%, 40% and 50%, 40% and 45%, 45% and 50% as compared to the level of IgE antibodies (e.g., total IgE antibodies or allergen-specific IgE antibodies) in the subject (or a sample thereof) prior to administration of the compositions. In some embodiments, administration of the compositions described herein results in levels of IgE antibodies (e.g., total IgE antibodies or allergen-specific IgE antibodies) that are reduced by between 30% and 50%, 30% and 45%, 35% and 45%, 30% and 40%, 35% and 40%, 40% and 50%, 40% and 45%, 45% and 50% as compared to the level of IgE antibodies in another subject (e.g., a reference subject) who did not receive the compositions.

The presence and/or quantity of IgE antibodies in a subject, including the presence and/or quantity of allergen-specific IgE antibodies, can be assessed by methods known in the art. For example, a sample, such as a blood or plasma sample, may be obtained from a subject and subjected to analysis, for example by immunoassays (e.g., radio allergosorbent test (RAST), fluorescent allergosorbant test (FAST), enzyme-linked immunosorbent assays (ELISA)) and protein arrays. The presence of allergen-specific IgE antibodies may, additionally or alternatively, be assessed using a skin test (e.g., skin prick test).

In some embodiments, the compositions and methods described herein (e.g., gel-based inulin formulations associated with one or more therapeutic agents (e.g., immunomodulatory agent, immunosuppressant, allergen)) (e.g., a gel-based inulin formulation associated with one or more therapeutic agents (e.g., immunomodulatory agent, immunosuppressant, allergen) and a prebiotic compound) suppress one or more Th2 immune responses. In some embodiments, the compositions and methods described herein suppress the development or differentiation of Th2 cells (also referred to as type 2 helper T cells). In some embodiments, the compositions and methods described herein suppress the activity of Th2 cells. As will be evident by one of ordinary skill in the art, Th2 cells are a subject of CD4+ cells that produce IL-4, IL-5, IL-6, IL-10, and/or IL-13 and may be involved in promoting IgE antibody responses and/or eosinophil activity. The differentiation of CD4+ cells to Th2 cells is promoted by the presence of IL-4 and/or IL-12 and activation of the transcription factors STAT6 and GATA3. In some embodiments, the amount of IgE antibodies may be assessed as a marker of Th2 immune responses in a subject.

In some embodiments, administration of the compositions described herein (e.g., gel-based inulin formulations associated with one or more therapeutic agents (e.g., immunomodulatory agent, immunosuppressant, allergen)) (e.g., a gel-based inulin formulation associated with one or more therapeutic agents (e.g., immunomodulatory agent, immunosuppressant, allergen) and a prebiotic compound) results in levels of Th2 immune responses that are reduced by at least 1.5-fold, 2-fold, 3-fold, 4-fold, 5-fold, 6-fold, 7-fold, 8-fold, 9-fold, 10-fold, 20-fold, 30-fold, 40-fold, 50-fold, 100-fold, 1000-fold, 10⁴-fold, 10⁵-fold or more, as compared to Th2 immune response in the subject (or a sample thereof) prior to administration of the compositions. In some embodiments, administration of the compositions described herein results in Th2 immune responses that are reduced by at least 1.5-fold, 2-fold, 3-fold, 4-fold, 5-fold, 6-fold, 7-fold, 8-fold, 9-fold, 10-fold, 20-fold, 30-fold, 40-fold, 50-fold, 100-fold, 1000-fold, 10⁴-fold, 10⁵-fold or more, as compared to Th2 immune responses in another subject (e.g., a reference subject) who did not receive the compositions.

In some embodiments, administration of the compositions described herein (e.g., gel-based inulin formulations associated with one or more therapeutic agents (e.g., immunomodulatory agent, immunosuppressant, allergen)) (e.g., a gel-based inulin formulation associated with one or more therapeutic agents (e.g., immunomodulatory agent, immunosuppressant, allergen) and a prebiotic compound) results in levels of Th2 immune responses that are reduced by at least 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 35%, 40%, 45%, 50%, 55%, 60%, 65%, 70%, 75%, 80%, 85%, 90%, 95%, or 100%, as compared to Th2 immune response in the subject (or a sample thereof) prior to administration of the compositions. In some embodiments, administration of the compositions described herein results in Th2 immune responses that are reduced by at least 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 35%, 40%, 45%, 50%, 55%, 60%, 65%, 70%, 75%, 80%, 85%, 90%, 95%, or 100%, as compared to Th2 immune responses in another subject (e.g., a reference subject) who did not receive the compositions.

The presence or level of a Th2 immune response may be assessed using any method known in the art. The presence or level of a Th2 immune response may be assessed, for example, by detecting and/or quantifying the number of Th2 cells in a sample obtained from the subject, such as by detecting a cellular marker indicative of the Th2 cells; assessing transcription profile associated with Th2 cells; assessing a direct or indirect activity of Th2 cells; and/or by measuring the production of one or more cytokines produced by Th2 cells (e.g., IL-4, IL-5, IL-6, IL-10, IL-13).

In some embodiments, administration of the compositions provided herein (e.g., gel-based inulin formulations associated with one or more therapeutic agents (e.g., immunomodulatory agent, immunosuppressant, allergen)) (e.g., a gel-based inulin formulation associated with one or more therapeutic agents (e.g., immunomodulatory agent, immunosuppressant, allergen) and a prebiotic compound) results in a healthy microbiome that modulates an immune response associated with allergy (e.g., a food allergy) in a subject. In some embodiments, administration of the compositions provided herein results in a healthy microbiome that modulates an immune response associated with allergy (e.g., a food allergy) in a subject. In some embodiments, administration of the compositions provided herein results in a healthy microbiome that induces the accumulation and/or proliferation of regulatory T cells in a subject. In some embodiments, administration of the compositions provided herein results in a healthy microbiome that suppresses production of IgE antibodies in a subject. In some embodiments, administration of the compositions provided herein results in a healthy microbiome that suppresses Th2 immune responses in a subject.

In some embodiments, the therapeutically effective amount of any of the compositions described herein (e.g., gel-based inulin formulations associated with one or more therapeutic agents (e.g., immunomodulatory agent, immunosuppressant, allergen)) (e.g., a gel-based inulin formulation associated with one or more therapeutic agents (e.g., immunomodulatory agent, immunosuppressant, allergen) and a prebiotic compound) is an amount sufficient to treat the allergy. In some embodiments, the therapeutically effective amount of any of the compositions described herein is an amount sufficient to reduce one or more symptom associated with the allergy. In some embodiments, the therapeutically effective amount of any of the compositions described herein is an amount sufficient to modulate one or more immune responses associated with allergy, such as a food allergy. For example, in some embodiments, the therapeutically effective amount of any of the compositions described herein is an amount sufficient to induce the proliferation and/or accumulation of regulatory T cells in the subject. In some embodiments, the therapeutically effective amount of any of the compositions described herein is an amount sufficient to suppress the production of IgE antibodies (e.g., total IgE antibodies or allergen-specific IgE antibodies). In some embodiments, the therapeutically effective amount of any of the compositions described herein is an amount sufficient to suppress one or more Th2 immune responses. In some embodiments, the therapeutically effective amount of any of the compositions described herein is an amount sufficient to allow a subject to survive a challenge with the allergen (e.g., in case of an anaphylactic allergic response in the inadvertent exposure to a peanut allergen).

In some embodiments, the compositions and methods described herein (e.g., gel-based inulin formulations associated with one or more therapeutic agents (e.g., immunomodulatory agent, immunosuppressant, allergen)) (e.g., a gel-based inulin formulation associated with one or more therapeutic agents (e.g., immunomodulatory agent, immunosuppressant, allergen) and a prebiotic compound) are used to treat autoimmune disorders (e.g., rheumatoid arthritis, multiple sclerosis, diabetes, etc). Examples of such conditions include, but are not limited to, rheumatoid arthritis, multiple sclerosis diabetes (e.g., type 1 diabetes mellitus), autoimmune diseases of the thyroid (e.g., Hashimoto’s thyroiditis, Graves’ disease), thyroid-associated ophthalmopathy and dermopathy, hypoparathyroidism, Addison’s disease, premature ovarian failure, autoimmune hypophysitis, pituitary autoimmune disease, immunogastritis, pernicious angemis, celiac disease, vitiligo, myasthenia gravis, pemphigus vulgaris and variants, bullous pemphigoid, dermatitis herpetiformis Duhring, epidermolysis bullosa acquisita, systemic sclerosis, mixed connective tissue disease, Sjogren’s syndrome, systemic lupus erythematosus, Goodpasture’s syndrome, rheumatic heart disease, autoimmune polyglandular syndrome type 1, Aicardi-Goutières syndrome, Acute pancreatitis Age-dependent macular degeneration, Alcoholic liver disease, Liver fibrosis, Metastasis, Myocardial infarction, Nonalcoholic steatohepatitis (NASH), Parkinson’s disease, Polyarthritis/fetal and neonatal anemia, Sepsis, and inflammatory bowel disease.

As used herein, the term “therapeutically effective amount” may be used interchangeably with the term “effective amount.” A therapeutically effective amount or an effective amount of a composition, such as a pharmaceutical composition, as described herein, is any amount that results in a desired response or outcome in a subject, such as those described herein, including but not limited to delay the manifestation, arrest the progression, relieve or alleviate at least one symptom of the disease that is treated using the methods described herein (e.g., allergy).

Any of the methods described herein may be for the treatment of allergy in a subject. As used herein, methods of treating allergy involve relieving or alleviating at least one symptom associated with the allergy, or slowing or preventing the onset of an allergic reaction upon contact or exposure to an allergen.

Also within the scope of the present disclosure are methods involving determining whether a subject has or is at risk of having an allergy or having an allergic reaction in response to an allergen. In some embodiments, if the subject is determined to have an allergy or be at risk for having an allergic reaction in response to an allergen, the subject is administered any of the compositions described herein (e.g., gel-based inulin formulations associated with one or more allergens) (e.g., a gel-based inulin formulation associated with one or more allergens and one or both of a prebiotic compound and a therapeutic agent). Methods of determining whether a subject has or is at risk of an allergy or having an allergic reaction in response to an allergen are known in the art and include, for example, detecting the presence or a level of IgE antibodies (e.g., total IgE antibodies, allergen-specific IgE antibodies), detecting the presence or a level of one or more Th2 immune response, or performing an allergy skin test. In some embodiments, the methods involve assessing whether the subject has or is at risk of having a food allergy. In some embodiments, if the subject is determined to have a food allergy or be at risk for having an allergic reaction in response to a food allergen, the subject is administered any of the compositions containing the bacterial strains described herein.

Any of the compositions described herein (e.g., gel-based inulin formulations associated with one or more therapeutic agents (e.g., immunomodulatory agent, immunosuppressant, allergen)) (e.g., a gel-based inulin formulation associated with one or more therapeutic agents (e.g., immunomodulatory agent, immunosuppressant, allergen) and a prebiotic compound) are provided in any form, for example in an aqueous form, such as a solution or a suspension, embedded in a semi-solid form, in a powdered form, or freeze-dried form. In some embodiments, the compositions are lyophilized. Methods of lyophilizing compositions are well known in the art.

In some embodiments, the compositions further comprise an acceptable excipient. An “acceptable” excipient refers to an excipient that must be compatible with the active ingredient and not deleterious to the subject to which it is administered. In some embodiments, the pharmaceutically acceptable excipient is selected based on the intended route of administration of the composition, for example a composition for oral or nasal administration may comprise a different pharmaceutically acceptable excipient than a composition for rectal administration. Examples of excipients include sterile water, physiological saline, solvent, a base material, an emulsifier, a suspending agent, a surfactant, a stabilizer, a flavoring agent, an aromatic, an excipient, a vehicle, a preservative, a binder, a diluent, a tonicity adjusting agent, a soothing agent, a bulking agent, a disintegrating agent, a buffer agent, a coating agent, a lubricant, a colorant, a sweetener, a thickening agent, and a solubilizer.

Pharmaceutical compositions of the invention can be prepared in accordance with methods well known and routinely practiced in the art. The pharmaceutical compositions described herein may further comprise any carriers or stabilizers in the form of a lyophilized formulation or an aqueous solution. Acceptable excipients, carriers, or stabilizers may include, for example, buffers, antioxidants, preservatives, polymers, chelating reagents, and/or surfactants. Pharmaceutical compositions are preferably manufactured under GMP conditions. The pharmaceutical compositions can be used orally, nasally or parenterally, for instance, in the form of capsules, tablets, pills, sachets, liquids, powders, granules, fine granules, film-coated preparations, pellets, troches, sublingual preparations, chewables, buccal preparations, pastes, syrups, suspensions, elixirs, emulsions, liniments, ointments, plasters, cataplasms, transdermal absorption systems, lotions, inhalations, aerosols, injections, suppositories, and the like. In some embodiments, the pharmaceutical compositions can be used by injection, such as by intravenous, intramuscular, subcutaneous, or intradermal administration.

Also within the scope of the present disclosure are pharmaceutical compositions for administration by additional or alternative routes. In some embodiments, the pharmaceutical compositions are formulated for sublingual administration. In some embodiments, the pharmaceutical compositions are formulated for administration by injection.

In some embodiments, the pharmaceutical composition includes an adjuvant associated with providing a benefit in the treatment of allergy. In some embodiments, the pharmaceutical composition includes one or more components of an oral immunotherapeutic, an epicutaneous immunotherapeutic, or a sublingual immunotherapeutic.

In some embodiments, the compositions described herein are formulated into pharmaceutically acceptable dosage forms by conventional methods known to those of skill in the art. Dosage regimens are adjusted to provide the optimum desired response (e.g., the prophylactic or therapeutic effect). In some embodiments, the dosage form of the composition is a tablet, pill, capsule, powder, granules, solution, or suppository. In some embodiments, the pharmaceutical composition is formulated for oral administration. In some embodiments, the pharmaceutical composition is formulated for rectal administration, e.g., as a suppository. In some embodiments, the pharmaceutical composition is formulated for delivery to the intestine or a specific area of the intestine (e.g., the colon) by providing an appropriate coating (e.g., a pH specific coating, a coating that can be degraded by target area specific enzymes, or a coating that can bind to receptors that are present in a target area). In some embodiments, the composition is a sugar-coated tablet, gel capsule, gel, emulsion, tablet, wafer capsule, hydrogel, nanofiber gel, electrospun fiber, food bar, confectionery, fermented milk, fermented cheese, chewing gum, powder or toothpaste.

Dosages of the active ingredients in the pharmaceutical compositions of the present invention can be varied so as to obtain an amount of the active ingredient which is effective to achieve the desired pharmaceutical response for a particular subject, composition, and mode of administration, without being toxic or having an adverse effect on the subject. The selected dosage level depends upon a variety of factors including the activity of the particular compositions of the present invention employed, the route of administration, the time of administration, the duration of the treatment, other drugs, compounds and/or materials used in combination with the particular compositions employed, the age, sex, weight, condition, general health and prior medical history of the subject being treated, and like factors.

A physician, veterinarian or other trained practitioner, can start doses of the pharmaceutical composition at levels lower than that required to achieve the desired therapeutic effect and gradually increase the dosage until the desired effect (e.g., treatment of allergy, modulation of one or more immune responses associated with allergy) is achieved. In general, effective doses of the compositions of the present invention, for the prophylactic treatment of groups of people as described herein vary depending upon many different factors, including routes of administration, physiological state of the subject, whether the subject is human or an animal, other medications administered, and the therapeutic effect desired. Dosages need to be titrated to optimize safety and efficacy.

In some embodiments, the dosing regimen entails oral administration of a dose of any of the compositions described herein. In some embodiments, the dosing regimen entails oral administration of multiple doses of any of the compositions described herein. In some embodiments, any of the compositions described herein are administered the subject once, twice, 3 times, 4 times, 5 times, 6 times, 7 times, 8 times, 9 times, or at least 10 times, or more. In some embodiments, any of the compositions described herein are administered the subject in multiple doses at a regular interval, such as every 2 weeks, every month, every 2 months, every 3 months, every 4 months, every 5 months, every 6 months, or more. In some embodiments, one dose of any of the compositions described herein is administered and a second dose of the composition is administered the following day (e.g., consecutive day). In some embodiments, one dose of any of the compositions described herein is administered and each of the additional doses of the composition are administered on consecutive days (e.g., first dose on day 1, second dose of day 2, third dose on day 3, etc.).

In one aspect, the disclosure provides methods comprising administration of multiple daily doses of the pharmaceutical compositions. In some embodiments, the pharmaceutical compositions are administered on a daily basis for 2 days, 3 days, 4, days, 5, days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 1 month, 2 months, 3 months, 4 months, 5 months, 6 months, 7 months, 8 months, 9 months, 10 months, 11 months, 12 months or more.

In some embodiments, the disclosure provides methods comprising administration of one or more doses of the pharmaceutical compositions to a subject, determining if the subject is responding to the administration of the one or more doses of the pharmaceutical compositions, e.g., by measuring the level of T regulatory cells, IgE cells or doing a skin test, wherein if the response is not associated with the desired effect (e.g., insufficient levels of T regulatory cell, or a strong response to a skin test), additional doses of the pharmaceutical compositions are administered.

Aspects of the present disclosure also provide food products comprising any of the compositions provided herein and a nutrient. Also with the scope of the present disclosure are food products comprising any of the bacterial strains described herein and a nutrient. Food products are, in general, intended for the consumption of a human or an animal. Any of the compositions described herein may be formulated as a food product. In some embodiments, the bacterial strains are formulated as a food product in spore form. In some embodiments, the bacterial strains are formulated as a food product in vegetative form. In some embodiments, the food product comprises both vegetative bacteria and bacteria in spore form. The compositions disclosed herein can be used in a food or beverage, such as a health food or beverage, a food or beverage for infants, a food or beverage for pregnant women, athletes, senior citizens or other specified group, a functional food, a beverage, a food or beverage for specified health use, a dietary supplement, a food or beverage for patients, or an animal feed.

Non-limiting examples of the foods and beverages include various beverages such as juices, refreshing beverages, tea beverages, drink preparations, jelly beverages, and functional beverages; alcoholic beverages such as beers; carbohydrate-containing foods such as rice food products, noodles, breads, and pastas; paste products such as fish hams, sausages, paste products of seafood; retort pouch products such as curries, food dressed with a thick starchy sauces, soups; dairy products such as milk, dairy beverages, ice creams, cheeses, and yogurts; fermented products such as fermented soybean pastes, yogurts, fermented beverages, and pickles; bean products; various confectionery products such as Western confectionery products including biscuits, cookies, and the like, Japanese confectionery products including steamed bean-jam buns, soft adzuki-bean jellies, and the like, candies, chewing gums, gummies, cold desserts including jellies, cream caramels, and frozen desserts; instant foods such as instant soups and instant soy-bean soups; microwavable foods; and the like. Further, the examples also include health foods and beverages prepared in the forms of powders, granules, tablets, capsules, liquids, pastes, and jellies.

In some embodiments, any of the methods described herein may further comprise administering (e.g., simultaneously or at different times) additional therapeutic agents. Examples of such therapeutic agents include, but are not limited to, disease-modifying antirheumatic drugs (e.g., leflunomide, methotrexate, sulfasalazine, hydroxychloroquine), biologic agents (e.g., rituximab, infliximab, etanercept, adalimumab, golimumab), nonsteroidal anti-inflammatory drugs (e.g., ibuprofen, celecoxib, ketoprofen, naproxen, piroxicam, diclofenac), analgesics (e.g., acetaminophen, tramadol), immunomodulators (e.g., anakinra, abatacept), glucocorticoids (e.g., prednisone, methylprednisone), TNF-α inhibitors (e.g., adalimumab, certolizumab pegol, etanercept, golimumab, infliximab), IL-1 inhibitors, and metalloprotease inhibitors. In some embodiments, the therapeutic agents include, but are not limited to, infliximab, adalimumab, etanercept, or parenteral gold or oral gold. In some instances, the therapeutic agent is an immunomodulatory agent or immunosuppressant (e.g., statins; mTOR inhibitors, such as rapamycin or a rapamycin analog; TGF-β signaling agents; TGF-β receptor agonists; histone deacetylase inhibitors, such as Trichostatin A; corticosteroids; inhibitors of mitochondrial function, such as rotenone; P38 inhibitors; NF-κβ inhibitors, such as 6Bio, Dexamethasone, TCPA-1, IKK VII; adenosine receptor agonists; prostaglandin E2 agonists (PGE2), such as Misoprostol; phosphodiesterase inhibitors, such as phosphodiesterase 4 inhibitor (PDE4), such as Rolipram; proteasome inhibitors; kinase inhibitors; G-protein coupled receptor agonists; G-protein coupled receptor antagonists; glucocorticoids; retinoids; cytokine inhibitors; cytokine receptor inhibitors; cytokine receptor activators; peroxisome proliferator-activated receptor antagonists; peroxisome proliferator-activated receptor agonists; histone deacetylase inhibitors; calcineurin inhibitors; phosphatase inhibitors; PI3 KB inhibitors, such as TGX-221; autophagy inhibitors, such as 3-Methyladenine; aryl hydrocarbon receptor inhibitors; proteasome inhibitor I (PSI); and oxidized ATPs, such as P2X receptor blockers. Immunosuppressants also include IDO, vitamin D3, cyclosporins, such as cyclosporine A, aryl hydrocarbon receptor inhibitors, resveratrol, azathiopurine (Aza), 6-mercaptopurine (6-MP), 6-thioguanine (6-TG), FK506, sanglifehrin A, salmeterol, mycophenolate mofetil (MMF), aspirin and other COX inhibitors, niflumic acid, estriol, triptolide; OPN-305, OPN-401; Eritoran (E5564); TAK-242; Cpn10; NI-0101; 1A6; AV411; IRS-954 (DV-1079); IMO-3100; CPG-52363; CPG-52364; OPN-305; ATNC05; NI-0101; IMO-8400; Hydroxychloroquine; CU-CPT22; C29; Ortho-vanillin; SSL3 protein; OPN-305; 5 SsnB; Vizantin; (+)-N-phenethylnoroxymorphone; VB3323; Monosaccharide 3; (+)-Naltrexone and (+)-naloxone; HT52; HTB2; Compound 4a; CNTO2424; TH1020; INH-ODN; E6446; AT791; CpG ODN 2088; ODN TTAGGG; COV08-0064; 2R9; GpG oligonucleotides; 2-aminopurine; Amlexanox; Bay11-7082; BX795; CH-223191; Chloroquine; CLI-095; CU-CPT9a; Cyclosporin A; CTY387; Gefitnib; Glybenclamide; H-89; H-131; Isoliquiritigenin; MCC950; MRT67307; OxPAPC; Parthenolide; Pepinh-MYD; Pepinh-TRIF; Polymyxin B; R406; RU.521; VX-765; YM201636; Z-VAD-FMK; and AHR-specific ligands; including but not limited to 2,3,7,8-tetrachloro-dibenzo-p-dioxin (TCDD); tryptamine (TA); and 6 formylindolo[3,2 b]carbazole (FICZ)). In particular embodiments, the immunosuppressant is fingolimod; 2-(1′H-indole-3′-carbonyl)-thiazole-4-carboxylic acid methyl ester (ITE) or related ligands; Trichostatin A; and/or Suberoylanilide hydroxamic acid (SAHA).

### EXPERIMENTAL

The following examples are provided to demonstrate and further illustrate certain preferred embodiments of the present invention and are not to be construed as limiting the scope thereof. Use of the terms “we”, “I” and “our” refer to the inventors.

### Example I

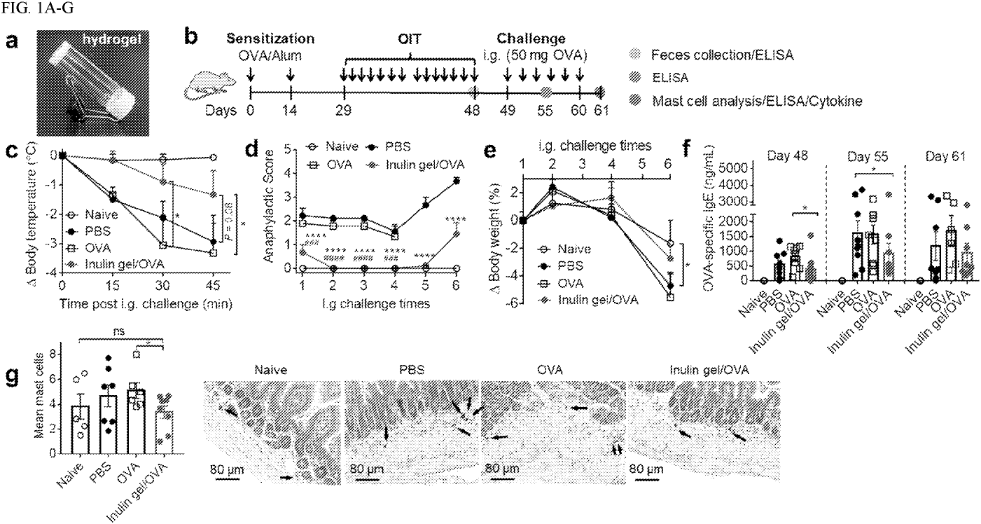

Inulin gel was prepared via a heating-cooling method (FIG. 1a). Mice were sensitized with intraperitoneal (i.p.) injection of albumin (OVA) and Alum, followed by OIT treatment and repeated oral challenge of OVA protein (FIG. 1b). After the 6^{th} OVA challenge, mice treated with inulin gel plus OVA exhibited markedly reduced core body temperature drop, compared with mice treated with PBS or free OVA (FIG. 1c). In addition, inulin gel plus OVA effectively decreased the anaphylactic response (FIG. 1d). Overall, inulin gel plus OVA effectively alleviated the diarrhea-induced body weight drop (FIG. 1e). We also observed that inulin gel plus OVA substantially reduced the OVA-specific IgE as well as the mast cells in the jejunum after OVA challenge (FIGS. 1f,g).

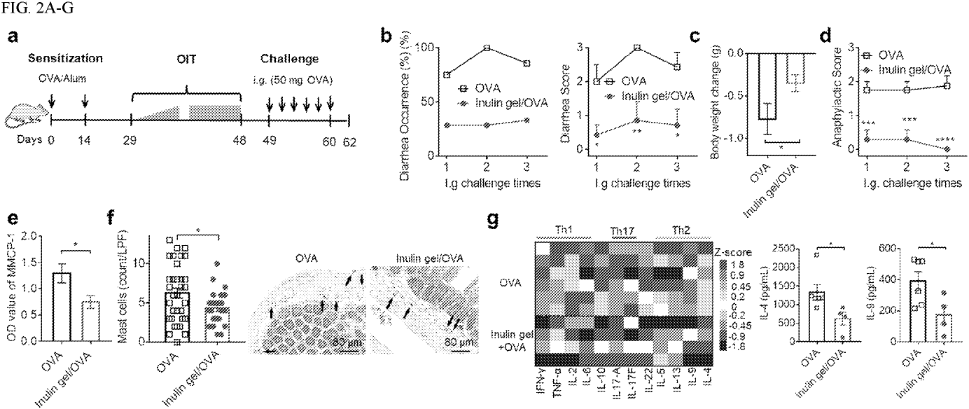

To simulate the widely used therapeutic plan in clinic, we next provided the escalated dose of OVA in OIT treatment (FIG. 2a). Some mice in the OVA group died after the 3^{rd} OVA challenge, so we only recorded the diarrhea as well as anaphylactic response up the 3^{rd} OVA challenge. Consistent with the results using low dose of OVA in OIT (FIG. 1), inulin gel plus escalated dose of OVA dramatically decreased the occurrence and severity of diarrhea, the anaphylactic score, and the body weight drop, compared with free OVA (FIGS. 2b-d). These results indicate that inulin gel could avoid the potential adverse events associated with unformulated OVA allergen in this model. Inulin gel also reduced the mucosal mast cell protease-1 (MMCP-1), the mast cells in jejunum, and skewed the Th2 cytokines to Th1/Th17 cytokines in the survivors (FIGS. 2e-g).

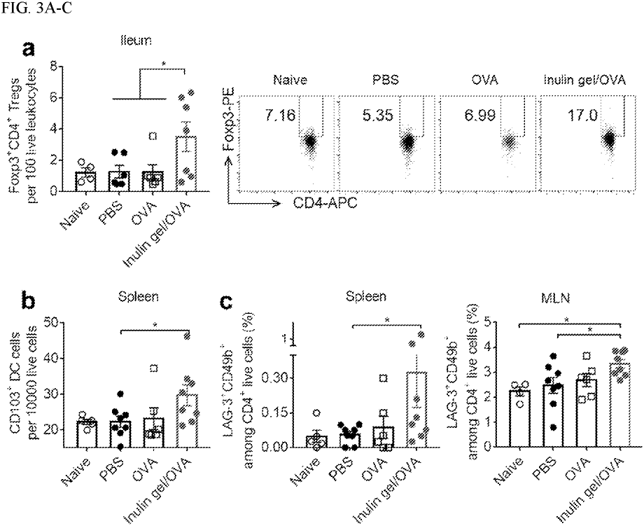

We next analyzed the ileum, spleen, and mesenteric lymph nodes (MLN) and observed that inulin gel plus OVA induced higher frequencies of Foxp3⁺CD4⁺ Tregs in ileum, CD103⁺ DC cells in spleen, and the LAG3⁺CD49b⁺ Tr1 cells in spleen and MLN (FIG. 3). As these cells are associated with immune tolerance, these results suggest that inulin gel serves as a platform for delivering allergen (or autoantigen) and inducing immune tolerance.

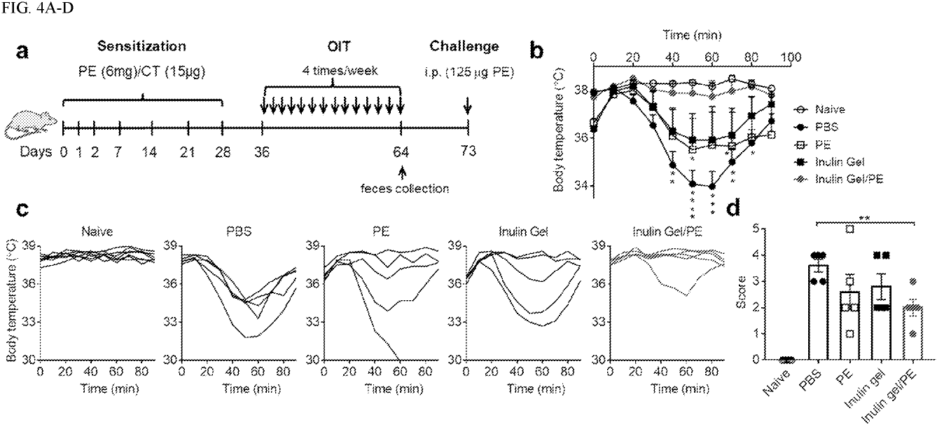

Finally, we established a peanut allergy model via orally administered cholera toxin (CT) and peanut extract (PE). OIT with inulin gel plus PE effectively protected mice against PE challenge, as shown by negligible body temperature drop and reduced anaphylactic response (FIG. 4). These results demonstrate the utility of inulin gel delivering allergen (or autoantigen) in multiple allergy models.

### Materials and Methods

Preparation and characterization of inulin gel, PE or OVA loaded inulin gel. For OVA-related study, 495 mg of inulin from chicory (Sigma-Aldrich) was dissolved in 1.35 mL PBS and heated for 5 min. OVA (Sigma-Aldrich) was added right after cool-down of inulin. For PE-related study, 300 mg of inulin from chicory (Sigma-Aldrich) was dissolved in 0.9 mL deionized water, after heating for 5 min, 8 mg of PE was added right after cool-down. Samples were kept at 4° C. for 24 h. For peanut extract preparation, 40 g of raw peanut powder was added to 500 mL of DI water and stirred for 2 h at room temperature, followed by sonication for 15 min. The suspension was centrifuged at 3000xg for 30 min. Then the supernatant was carefully collected and further centrifuged at 8000xg for 60 min. The supernatant containing the protein was aliquoted and lyophilized. The content of protein in peanut extraction (around 20%) was determined by BCA assay.

In vivo oral sensitization, immunotherapy, and challenge. Animals were cared for following the federal, state, and local guidelines. All work conducted on animals was in accordance with and approved by the Institutional Animal Care and Use Committee (IACUC). Female C3H/HeJ mice or BABL/c (5-6-week-old) from Jackson Laboratory were maintained on the normal mouse chow diet (PicoLab® Laboratory Rodent Diet 5L0D*). Mice were acclimatization for one week before experiment. For alum/OVA model, BABL/c mice were sensitized Intraperitoneally (i.p.) with PBS (sham-sensitization, naïve group) or OVA (50 µg/dose) and alum (1 mg/dose, Sigma-Aldrich) in 150 µL PBS on days 1 and 14. From day 29, these sensitized mice were orally administered with PBS, OVA (1 mg/dose), inulin gel (55 mg/dose) or inulin gel (55 mg/dose) plus OVA (1 mg/dose). In the escalated dose of OVA setting, mice were first orally gavaged with OVA or inulin gel plus OVA using 0.25, 0.5, 1, 2, 4, 8, 12, 16 mg heated OVA (heating under 70° C. for 2 min), following with 20 mg of heated OVA for 6 days. From day 49, the mice were intragastrically (i.g.) challenged with 50 mg of OVA in 250 µL PBS every other day for a total of 6 gavages. Before i.g. challenging, mice were deprived of food for 4 h. Body weight before all times of i.g. challenge were recorded, while the body temperature in the last time of i.g. challenge was measured. For PE/CT sensitization model, mice were randomly allocated, and i.g. sensitized with PBS (sham-sensitization, naïve group) or PE (6 mg/dose) and cholera toxin (CT, as an adjuvant, 10 µg/dose) in 200 µL PBS on days 1, 7, 14, 21, and 28. From day 36, these mice were orally administered with PBS, PE, inulin gel (45 mg/dose) or inulin gel (45 mg/dose) plus PE (1.2 mg per dose) 4 times per week, respectively. Naïve mice were orally administrated with PBS as a control. On day 73, mice were i.p. injected with 125 µg PE and the body temperature was recorded at preset times via rectal temperature probe.

Assessment of hypersensitivity reactions and diarrhea. Anaphylactic symptoms were evaluated between 30 to 50 minutes after i.p. challenge of PE (125 µg/dose) or i.g. challenge of OVA (50 mg/dose). Anaphylactic symptoms were scored via visually observation: 0, no symptoms; 1, scratching and rubbing around the nose and head; 2, puffiness around the eyes and mouth, pilar erection, reduced activity, and/or decreased activity with increased respiratory rate; 3, wheezing, labored respiration, and cyanosis around the mouth and the tail; 4, no activity after prodding or tremor and convulsion; and 5, death or the body temperature below 30° C. For OVA study, mice with profuse liquid stool up to 1 h after i.g. challenge were recorded as diarrhea-positive animals. The clinical diarrhea score was assessed via visually observation: 0, normal; 1, soft; 2, running; 3, liquid; 4, bloody.

OVA specific antibody detection. Serum samples were collected on days finishing OIT treatment, 1 week after i.g. challenge, and 24th h after last time of i.g. challenge with 50 mg of OVA, respectively. OVA-specific antibody in serum was detected via ELISA method. Briefly, the 96-well ELISA plate was coated with 10 µg of OVA per well and incubated overnight at 4° C. The plate was washed and blocked with 1% BSA PBS buffer for 2 h. Serum samples or standard mouse anti-ovalbumin (isotype IgE, Bio-rad, USA) diluted to preset fold were incubated in 96-well plate for 1 h. After repeated washing, HRP-conjugated goat anti-mouse IgE (1:4000 dilution, SouthernBiotech) was added to each well and incubated for 1 h. The plate was thoroughly washed and 100 µL of TMB solution was added to each well. 10 min later, the stop solution (1 M H₂SO₄) was added and the optical density (OD) value at 450 nm was detected via a microplate reader.

Cytokine expression. 24 h after the last time i.g. challenge, the spleens from various groups were obtained aseptically. The splenocytes were cultured in 96-well plates (1 × 10⁶ cells per well) and re-stimulated with OVA (250 µg/mL). After 72 h incubation, the supernatant were obtained and the cytokines were tested via LEGENDplex™ MU Th Cytokine Panel (12-plex) w/ VbP V03 (Biolegend, USA) and the data were analyzed by LEGENDplex™ Clound-Based Software.

In vivo immunological analyses of intestinal tissue and spleen. 24 h after the last time i.g. challenge, spleen, MLN, and ileum (1 cm distal of the cecum) samples were collected. MLN were isolated, ground and filtered through a 70-µm strainer. Spleens were ground, incubated with ACK lysis and then filtered through a 70-µm strainer. For ileum tissues, the tissue were cut into 1 cm pieces and treated with PBS containing 2% FACS buffer, 1.5 mM DTT, and 10 mM EDTA at 37° C. for 30 min with stirring to remove mucous and epithelial cells. The tissues were then minced and incubated with collagenase, DNase I (100 mg/ml), 5 mM MgCl2, 5 mM CaCl2, 5 mM HEPES, and 10% FBS with constant stirring at 37° C. for 45 min. Then the cell suspension were filtered through a 70-µm strainer. Cells were first stained with LIVE/DEAD fixable efluor 450. Cells were incubated with anti-CD16/CD32 (clone 2.4G2,BDBiosciences), then stained with fluorescent-labeled antibodies and stored (4° C. in the dark) until measurement.

Intestinal mast cell quantification. 24 h after the last i.g. challenge of OVA, jejunum tissue was collected 7-10 cm distal to the stomach and fixed in 4% formalin. The tissue was stained with Toluidine Blue via the In-Vivo Animal Core of the University of Michigan. At least 3 random sections per mouse were analyzed, and Toluidine Blue-positive stained cells was counted from around 3.5 mm length of tissue (magnification 20×).

### Example II

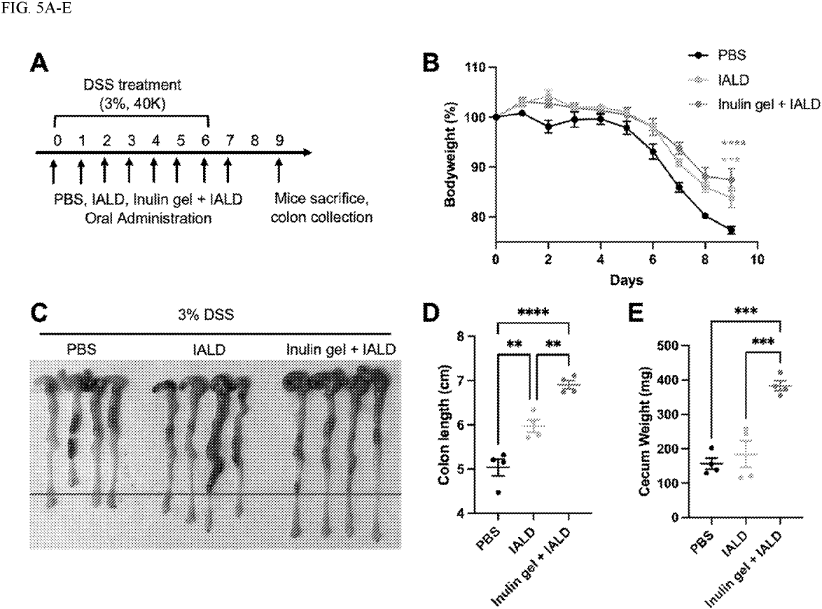

We examined whether oral administration of inulin gel loaded with an immune tolerizing drug can exert therapeutic efficacy against colitis. Mice were given 3% DSS in drinking water for 6 days to induce acute colitis. Starting day 0, mice were also orally gavaged with either PBS, an Ahr ligand, indole-3-aldehyde (IALD), or inulin gel formulated with IALD (inulin gel + IALD) (FIG. 5A). Treatment with inulin gel + IALD protected mice against severe bodyweight loss and shortening of the colon length (FIGS. 5B,C). In contrast, mice treated with PBS or IALD showed significant bodyweight loss and shortening of the colon length. Notably, mice treated with inulin gel + IALD had the highest cecum weight, which may be attributed to the microbiota-normalizing effect of inulin gel and immunoregulatory role of the Ahr ligands (see, K. Han, J. Nam, J. Xu, X. Sun, X. Huang, O. Animasahun, A. Achreja, J.H. Jeon, B. Pursley, N. Kamada, G.Y. Chen, D. Nagrath, J.J. Moon, Generation of systemic antitumour immunity via the in situ modulation of the gut microbiome by an orally administered inulin gel, Nature Biomedical Engineering, (2021); B. Stockinger, K. Shah, E. Wincent, AHR in the intestinal microenvironment: safeguarding barrier function, Nature Reviews Gastroenterology & Hepatology, 18 (2021) 559-570). These results indicate that inulin gel can be used to deliver immune-tolerizing agents to ameliorate colitis and other inflammatory bowel diseases.

Method: Six-week-old female C57BL/6 mice were acclimatized for 2 weeks before the start of the study. To induce colitis, mice were given 3% DSS (40 kDa; Alfa Aesar) supplemented in the drinking water for 6 days, followed by giving normal water. Inulin gel + IALD, IALD or PBS was orally gavaged into mice on predetermined days. The inulin gel + IALD was prepared by dissolving 300 mg of inulin from chicory (Sigma-Aldrich) in 0.9 ml deionized water and 0.1 ml IALD solution (50 mg/mL, DMSO). The inulin solution was thoroughly mixed with IALD solution by vortex and was heated to 70° C. for 5 min. The formulation was kept at room temperature for 12 h for gelation. The IALD was prepared similarly without adding the inulin. Bodyweight were measured daily over the experimental period. On day 9, mice were sacrificed, and the entire colon was excised. Colon length and the cecum weight was measured.

### INCORPORATION BY REFERENCE

The entire disclosure of each of the patent documents and scientific articles referred to herein is incorporated by reference for all purposes.

### EQUIVALENTS

The invention may be embodied in other specific forms without departing from the spirit or essential characteristics thereof. The foregoing embodiments are therefore to be considered in all respects illustrative rather than limiting the invention described herein. Scope of the invention is thus indicated by the appended claims rather than by the foregoing description, and all changes that come within the meaning and range of equivalency of the claims are intended to be embraced therein.

What is claimed is:

1. A composition comprising a gel-based inulin formulation associated with one or more therapeutic agents, wherein the one or more therapeutic agents are selected from an allergen, immunomodulatory agent, and an immunosuppressant.

2. The composition of claim 1, wherein associated with comprises one or more of the following: complexed, conjugated, encapsulated, absorbed, adsorbed, and admixed.

3. The composition of claim 1, wherein the gel-based inulin formulation has an average degree of polymerization at or higher than 20 and at or less than 47.

4. The composition of claim 1, wherein the gel-based inulin formulation has an average degree of polymerization of approximately 26.

5. The composition of claim 1, wherein the gel-based inulin formulation further comprises one or more prebiotic compounds.

6. The composition of claim 5, wherein the one or more prebiotic compounds are independently selected from the group consisting of a fructo-oligosaccharide, a short-chain fructo-oligosaccharide, an isomalt-oligosaccharide, a transgalacto-oligosaccharide, a pectin, a xylo-oligosaccharide, a chitosan-oligosaccharide, a beta-glucan, an arable gum modified starch, a resistant potato starch, guar gum, bean gum, gelatin, glycerol, a polydextrose, a D-tagatose, an acacia fiber, carob, an oat, and a citrus fiber.

7. The composition of claim 1, wherein upon administration in a therapeutically effective amount to a subject the composition is capable of one or more of:
treating an allergy (e.g., a food allergy),
reducing one or more symptom associated with an allergy (e.g., food allergy),
modulating one or more immune responses associated with an allergy (e.g., food allergy),
inducing the proliferation and/or accumulation of regulatory T cells in the subject,
suppressing the production of IgE antibodies (e.g., total IgE antibodies or allergen-specific IgE antibodies),
suppressing one or more Th2 immune responses, and
allowing the subject to survive a challenge with the allergen (e.g., in case of an anaphylactic allergic response in the inadvertent exposure to a peanut allergen).

8. The composition of claim 1, wherein the one or more allergens is independently selected from an allergen source selected from animal products, plants, food, insect stings, drugs, fungal spores, and microorganisms.

9. The composition of claim 1, wherein the one or more allergens are independently selected from an animal product allergen, plant allergens, insect sting allergens, drug allergens, fungal allergens, microorganism allergens.

10. The composition of claim 1, wherein the one or more allergens are independently selected from abalone (perlemoen), acerola, Alaska pollock, almond, aniseed, apple, apricot, avocado, banana, barley, bell pepper, Brazil nut, buckwheat, cabbage, carp, carrot, cashew, caster bean, celery, celeriac, cherry, chestnut, chickpea (garbanzo, bengal gram), cococa, coconut, cod, cotton seed, courgett (zucchini), crab, date, egg, fig, fish, flax seed (linseed), frog, garden plum, garlic, grape, hazelnut, kiwi fruit (Chinese gooseberry), lentil, lettuce, lobster, lupin (lupine), lychee, mackerel, maize (com), mango, melon, milk, mustard, oat oyster, peach, peanut (ground nuts, monkey nuts), pear, pecan, persimmon, pine nut, pineapple, pomegranate, poppy seed, potato, pumpkin, rice, rye, salmon, sesame, sesame seed, shrimp (black tiger shrimp, brown shrimp, greasyback shrimp, Indian prawn, Neptune rose shrimp, white shrimp), snail, soybean (soya), squid, strawberry, sunflower seed, tomato, tuna, turnip, walnut, and wheat (bread-making wheat, pasta wheat, kamut, spelt).

11. The composition of claim 1, wherein the one or more allergens are independently selected from
animal products including fur, dander, cockroach calyx, wool, dust mite excretion, and fel d 1 (e.g., a protein produced in cat saliva and sebaceous glands);
allergens from plant include plant pollens from grass such as ryegrass; weeds such as ragweed, nettle, sorrel; and trees such as birch, alder, hazel, oak, elm, and maple;
allergens from insect stings include bee sting, wasp sting, and mosquito stings;
drug allergens including penicillin, sulfonamides, quinidine, phenylbutazone, thiouracils, methyldopa, hydantoins, and salicytates;
fungal allergens include basidiospores such as Ganoderma; mushroom spores; allergens from the aspergillus and alternaria-penicillin families; and cladosporium spores;
allergens from microorganisms that can cause an allergic reaction include viruses and bacteria; and
food allergens including peanuts, tree nuts such as pecans and almonds, eggs, milk, shellfish, fish, wheat and their derivative, soy and their derivatives.

12. The composition of claim 1, wherein one or more allergens is two or more allergens.

13. The composition of claim 1, wherein the immunosuppressant or immunomodulatory agent is selected from the group comprising fingolimod; 2-(1′H-indole-3′-carbonyl)-thiazole-4-carboxylic acid methyl ester (ITE) or related ligands; Trichostatin A; Suberoylanilide hydroxamic acid (SAHA); statins; mTOR inhibitors; TGF-β signaling agents; TGF-β receptor agonists; histone deacetylase inhibitors; corticosteroids; inhibitors of mitochondrial function; NF-ĸβ inhibitors; adenosine receptor agonists; prostaglandin E2 agonists (PGE2; phosphodiesterase inhibitors; proteasome inhibitors; kinase inhibitors; G-protein coupled receptor agonists; G-protein coupled receptor antagonists; glucocorticoids; retinoids; cytokine inhibitors; cytokine receptor inhibitors; cytokine receptor activators; peroxisome proliferator-activated receptor antagonists; peroxisome proliferator-activated receptor agonists; histone deacetylase inhibitors; calcineurin inhibitors; phosphatase inhibitors; PI3 KB inhibitors; autophagy inhibitors; aryl hydrocarbon receptor inhibitors; proteasome inhibitor I (PSI); oxidized ATPs IDO; vitamin D3; cyclosporins; aryl hydrocarbon receptor inhibitors; resveratrol; azathiopurine (Aza); 6-mercaptopurine (6-MP); 6-thioguanine (6-TG); FK506; sanglifehrin A; salmeterol; mycophenolate mofetil (MMF); aspirin and other COX inhibitors; niflumic acid; estriol; triptolide; OPN-305, OPN-401; Eritoran (E5564); TAK-242; Cpn10; NI-0101; 1A6; AV411; IRS-954 (DV-1079); IMO-3100; CPG-52363; CPG-52364; OPN-305; ATNC05; NI-0101; IMO-8400; Hydroxychloroquine; CU-CPT22; C29; Ortho-vanillin; SSL3 protein; OPN-305; 5 SsnB; Vizantin; (+)-N-phenethylnoroxymorphone; VB3323; Monosaccharide 3; (+)-Naltrexone and (+)-naloxone; HT52; HTB2; Compound 4a; CNTO2424; TH1020; INH-ODN; E6446; AT791; CpG ODN 2088; ODN TTAGGG; COV08-0064; 2R9; GpG oligonucleotides; 2-aminopurine; Amlexanox; Bay 11-7082; BX795; CH-223191; Chloroquine; CLI-095; CU-CPT9a; Cyclosporin A; CTY387; Gefitnib; Glybenclamide; H-89; H-131; Isoliquiritigenin; MCC950; MRT67307; OxPAPC; Parthenolide; Pepinh-MYD; Pepinh-TRIF; Polymyxin B; R406; RU.521; VX-765; YM201636; Z-VAD-FMK; and AHR-specific ligands; including but not limited to 2,3,7,8-tetrachloro-dibenzo-p-dioxin (TCDD); tryptamine (TA); and 6 formylindolo[3,2 b]carbazole (FICZ).

14. A method of treating an allergy, preventing development of an autoimmune disorder and/or ameliorating symptoms of an autoimmune disorder, comprising administering to a subject in need thereof a therapeutically effective amount of a composition described in claim 1, wherein the autoimmune disorder is one or more of the following: allergic asthma, allergic colitis, animal allergies, atopic allergies, hay fever, skin allergy, hives, atopic dermatitis, anaphylaxis, allergic rhinitis, drug or medicinal allergy, eczema (atopic dermatitis), food allergy, fungal allergy, insect allergy (including insect bite/venom allergies), mold allergies, plant allergies, and pollenosis.

15. The method of claim 14, wherein the subject is a mammalian subject.

16. The method of claim 14, wherein the subject is a human subject.

17. The method of claim 14, wherein the administering results in the suppression of an immune response associated with a food allergy.

18. The method of claim 14, wherein the administering results in the suppression of the production of IgE antibodies.

19. The method of claim 14, wherein the administering results in the suppression of a Th2 immune response.

20. The method of claim 14, wherein the food allergy is selected from the group consisting of a nut allergy, a fish allergy, a wheat allergy, a milk allergy, a peanut allergy, a tree nut allergy, a shellfish allergy, a soy allergy, a seed allergy, a sesame seed allergy, and an egg allergy.

21. The method of claim 14, wherein the food allergy is a peanut allergy.

22. The method of claim 14, wherein the composition is formulated for oral administration.

23. The method of claim 14, wherein the wherein the composition is a sugar-coated tablet, gel capsule, gel, emulsion, tablet, wafer capsule, hydrogel, nanofiber gel, electrospun fiber, food bar, confectionery, fermented milk, fermented cheese, chewing gum, powder or toothpaste.
